(19) United States
(12) Patent Application Publication
(10) Pub. No.: US 2026/0094709 A1
(43) Pub. Date: Apr. 2, 2026

(54) INTEGRATED MIXED REALITY VISUALIZATION FOR DIAGNOSTIC IMAGING AND DATA MAPPING

(71) Applicant: Stephen McNutt, Laredo, TX (US)

(72) Inventor: Stephen McNutt, Laredo, TX (US)

(21) Appl. No.: 18/900,604

(22) Filed: Sep. 27, 2024

(51) Int. Cl. G16H 50/20; G16H 30/40

(52) U.S. Cl. CPC G16H 50/20; G16H 30/40

(57) ABSTRACT

Approaches are described for facilitating remote ophthalmic examinations using three-dimensional (3D) imaging and mixed reality technology. A system obtains real-time or stored 3D data of a patient's eye, capturing detailed anatomical structures. The system analyzes the 3D data to identify specific regions of the eye, such as the cornea or retina, and retrieves corresponding diagnostic data and patient-specific clinical information. The 3D data, diagnostic metrics, and clinical records are integrated to generate an interactive visualization, which is presented through a mixed reality interface. The system allows healthcare professionals to manipulate diagnostic overlays, investigate flagged abnormalities, and adjust the visualization using gesture-based inputs. Machine learning models may be applied to detect potential abnormalities in the eye, while the system also determines stages of the examination based on changes in the anatomical structure of the eye.

### BACKGROUND

### Field of the Art

The systems and methods disclosed herein are related generally to data processing and visualization systems and more specifically to systems and methods for capturing, processing, and presenting 2D and 3D imaging data with contextual information in a mixed reality environment.

### Discussion of the State of the Art

Doctors face challenges in reviewing and analyzing large volumes of medical data during patient examinations. The process often requires manual searching for relevant contextual information, such as test results and imaging data, from various sources. This manual search process can be time-consuming and disrupt the examination.

There is also the problem of a lack of a unified system that integrates real-time examination video with corresponding contextual information in a format that is easily accessible during the examination. Currently, doctors may need to switch between multiple devices or screens to access this information, causing delays and making it difficult to maintain focus on the patient.

Existing solutions have included the use of separate medical software systems that allow doctors to access patient data, such as electronic health records (EHR), and view medical images on different devices or screens. However, these systems typically require manual searching and navigation, slowing down the process as doctors must shift focus between examining the patient and locating the relevant information.

Another solution has been the use of telemedicine platforms, which allow for remote video consultations. While these platforms enable real-time interaction with patients, they generally do not integrate contextual data, such as medical scans or test results, in a way that synchronizes with the live examination video. This lack of integration forces doctors to use multiple disjointed systems, making it difficult to quickly correlate data with the examination. These solutions do not fully streamline the examination process, as they require manual input and fail to provide a seamless connection between the real-time exam and the necessary data.

### SUMMARY

Systems and methods in accordance with the embodiments described herein overcome various deficiencies in existing approaches to real-time medical examination and data integration. In particular, various embodiments utilize three-dimensional (3D) imaging and mixed reality environments to capture, process, and present contextual data during ophthalmic examinations. These systems address the technical challenge of real-time data ingestion, processing, and presentation by integrating heterogeneous data from multiple sources into a unified, interactive display.

In one embodiment, the system captures 3D video data of a patient's eye during an examination. The system can apply computer vision algorithms to analyze the 3D video in real-time, identifying specific regions of the eye, such as the cornea or retina, that are under examination. Once the system identifies the region, it retrieves relevant contextual information from associated data stores, including diagnostic data, test results, and scans, to assist the healthcare professional in their evaluation.

In certain embodiments, the system presents this contextual information to the doctor in real-time via a virtual reality (VR) or augmented reality (AR) headset. The system integrates the retrieved data with the live 3D video of the eye, allowing the doctor to interact with both in a unified mixed reality interface. By automating this process, the system reduces the need for manual data searches and device switching, improving the workflow of the medical examination.

The system operates in a cloud-based environment, allowing remote access and collaboration between multiple users across different locations. The distributed processing environment ensures that the system can scale to meet the demands of high data volumes while providing real-time feedback and interaction for the medical professional.

In various embodiments, machine learning models are used to enhance the system's ability to analyze the 3D video data and associated contextual information. These models are trained on large datasets of annotated eye images and medical information. The trained models can identify anatomical structures, detect abnormalities, and determine specific stages of the eye examination based on changes in the anatomical structures over time. For example, the system may analyze changes in retinal thickness or optic nerve cupping, detecting early signs of glaucoma or macular degeneration. These machine learning models enhance the diagnostic process by flagging potential abnormalities in real-time, enabling doctors to make more informed decisions during the examination.

The system's components include 3D video capture, computer vision-based analysis, and automated retrieval and presentation of contextual data. The system ingests and processes non-standardized data formats, standardizing them into a format that can be used in real-time within a mixed reality environment. This process ensures that all necessary data is available during the examination without requiring the doctor to perform manual searches or switch between multiple devices.

In certain embodiments, the system determines the stage of the eye examination based on the anatomical structures identified in the 3D video. The stage determination allows the system to adjust how data is retrieved and presented, ensuring that the doctor receives the most relevant information for the current phase of the examination.

The system also enables user interaction via gesture-based input, allowing the doctor to manipulate diagnostic overlays or zoom into specific regions of the eye during the examination. This feature enhances the usability of the mixed reality interface, providing an intuitive way to navigate and interact with complex 3D data.

Advantageously, the system improves upon existing approaches to real-time examination by automating data integration tasks that would otherwise be performed manually. Unlike traditional methods where doctors manually correlate data from different sources, the disclosed system automates this process and presents the data in a format optimized for real-time use. The technical improvements include the system's ability to automate these tasks and ingest data in non-standardized formats, transforming it into a format that can be integrated and processed in real-time.

Additionally, the system employs landmark detection and region-based tagging techniques to map diagnostic measurements and clinical records to specific regions of the 3D video data. These techniques enhance the accuracy of data integration and presentation, ensuring that the doctor has immediate access to relevant diagnostic information based on the identified anatomical structures.

The machine learning models used by the system provide further technical improvements by dynamically adjusting as new data is captured. The models are operable to detect examination phases, flag abnormalities, and continuously learn from new data to refine their diagnostic capabilities. This ensures that the system improves its performance over time and can handle a wide variety of ophthalmic conditions with precision.

The disclosed system realizes improvements to both computer technology and the medical examination process. It addresses the technical challenge of real-time data ingestion and standardization from heterogeneous data sources, improving the functioning of the system by streamlining the process of correlating complex medical data. By transforming non-standardized data into a format optimized for real-time mixed reality environments, the system enhances the efficiency of medical professionals during examinations.

In certain embodiments, the system's machine learning models provide a further technical advancement by enabling automated detection of abnormalities in the 3D data. These models are trained on large datasets of eye images and medical information and can identify subtle changes in anatomical structures that may not be immediately apparent to the human eye. The system dynamically integrates these findings into the mixed reality interface, providing real-time feedback to the doctor.

Furthermore, the system operates within a cloud-based environment, which allows for remote collaboration, distributed processing, and multi-user access. This cloud architecture improves the scalability and accessibility of the examination process, enabling users to interact with the data in real-time from different locations.

The system improves a technical field—including, e.g., medical examination and diagnosis—by automating the integration of 3D video and contextual information. By performing tasks traditionally done manually, the system enhances the overall examination process and transforms how data is processed, analyzed, and presented in medical diagnostics.

Various other functions and advantages are described and suggested below as may be provided in accordance with the various embodiments.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1A illustrates an example environment in which aspects of the various embodiments can be implemented.

FIG. 1B illustrates the network architecture of a remote examination system in accordance with various embodiments.

FIG. 2 illustrates an example computing environment including an integration system in accordance with an exemplary embodiment.

FIG. 3 illustrates an example classification pipeline of a machine learning system that can be utilized in accordance with various embodiments.

FIG. 4 illustrates an example process for determining training data that can be utilized in accordance with various embodiments.

FIG. 5 illustrates an example process for training a model that can be utilized in accordance with various embodiments.

FIG. 6 illustrates an example system for interacting with content using a mixed reality device.

FIG. 7 illustrates an example view that can presented to a user in accordance with various embodiments.

FIG. 8 illustrates an example system in accordance with various embodiments.

FIG. 9 illustrates an exemplary process for integrated mixed reality visualization for diagnostic imaging and data mapping in accordance with various embodiments.

FIG. 10 illustrates an exemplary process for guiding visual data capture and treatment in an integrated mixed reality environment using computer vision techniques in accordance with various embodiments.

FIG. 11 illustrates one embodiment of the computing architecture that supports an embodiment of the inventive disclosure.

FIG. 12 illustrates components of a system architecture that supports an embodiment of the inventive disclosure.

FIG. 13 illustrates components of a computing device that supports an embodiment of the inventive disclosure.

FIG. 14 illustrates components of a computing device that supports an embodiment of the inventive disclosure.

### DETAILED DESCRIPTION

The embodiments described herein relate to systems and methods for capturing, processing, and presenting 3D imaging data alongside contextual information in a mixed reality environment. The system can automatically analyze 3D video data using computer vision algorithms and other appropriate techniques and retrieve relevant contextual information, such as medical scans and measurements, based on the specific region of the eye under examination. In various embodiments, the system can generate diagnostic information using trained machine learning models and present this information to users in real-time through a virtual reality (VR) or augmented reality (AR) interface. In certain embodiments, the system operates in a cloud-based environment, enabling remote access and multi-user collaboration, and is operable to process non-standardized data from multiple sources, transforming it into a unified format for efficient real-time analysis and display.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Conceptual Architecture

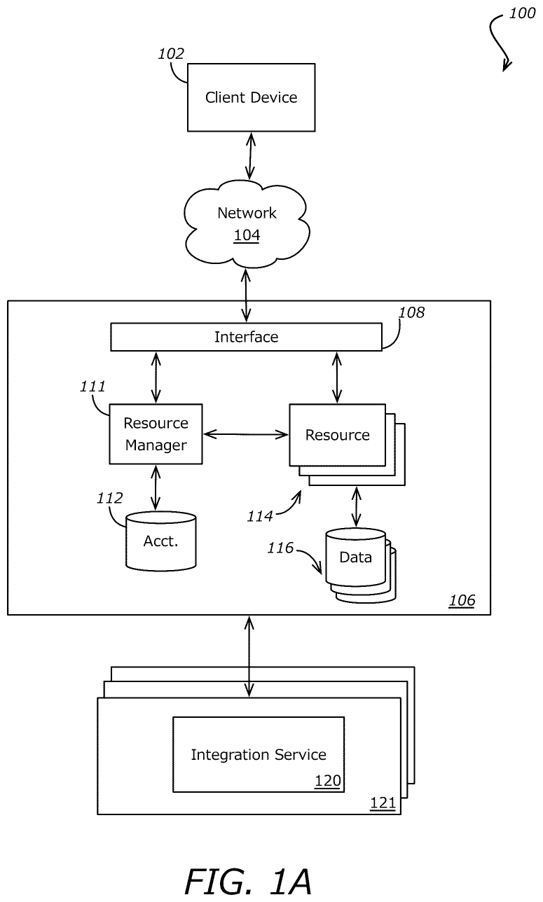

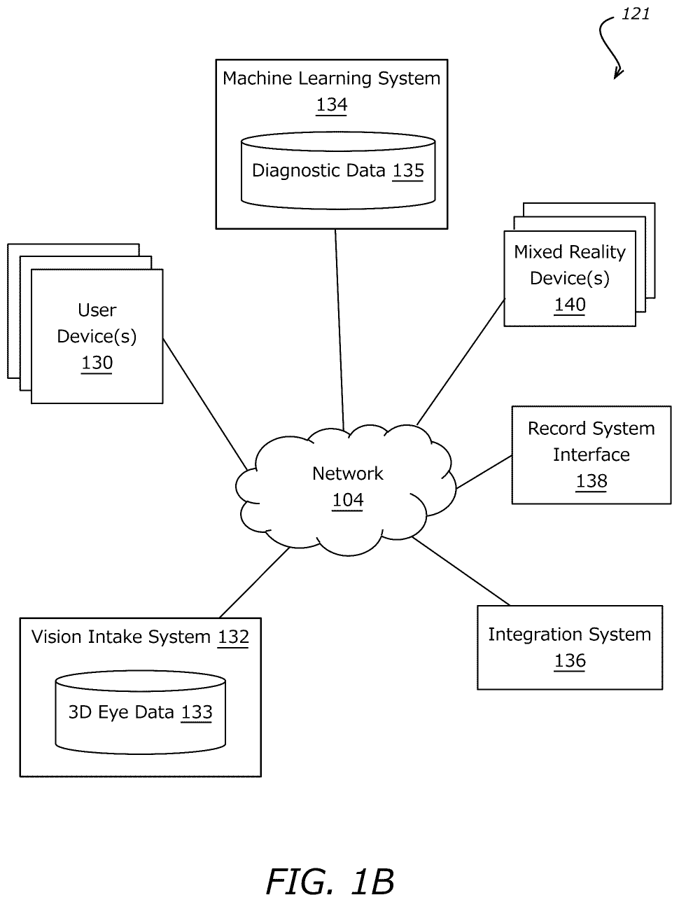

FIG. 1A illustrates an example environment 100 in which aspects of the various embodiments can be implemented. It should be understood that reference numbers are carried over between figures for similar components for purposes of simplicity of explanation, but such usage should not be construed as a limitation on the various embodiments unless otherwise stated.

In this example, a user can utilize a client device 102 to communicate across at least one network, such as network 104, with a resource provider environment 106. The client device 102 can include any appropriate electronic device operable to send and receive requests or other such information over the network 104, and convey information back to a user of the device. Examples of such client devices include personal computers, tablet computers, smartphones, notebook computers, and the like. The user can be a person authorized to manage or interact with aspects of the resource provider environment 106, such as medical professionals or system administrators.

The network 104 may include a variety of forms such as the Internet, cellular networks, or local area networks (LANs), and may allow for communication between the client device 102 and the resource provider environment 106. Communication can occur through both wired and wireless means, facilitating interaction within a cloud-based infrastructure that supports remote access to the system.

The resource provider environment 106 includes an integration service 120, which is configured to manage the communication and data exchanges between different components of the system. The integration service 120 can ingest data, such as 3D video data and contextual information, and ensure that this data is processed and delivered to the appropriate systems for analysis and display. The system operates in a cloud-based environment, allowing multiple instances of the integration service 120 to be activated for different users or medical professionals as needed.

In certain embodiments, the integration service 120 is designed to handle non-standardized data formats and convert them into a standardized format for real-time analysis and presentation. The integration service 120 is connected to various resources, such as application servers 114 and database servers 116, which manage the processing and storage of medical data, 3D video data, and associated contextual information.

The resource manager 111 in the resource provider environment 106 is responsible for managing user accounts and information, provisioning resources, and ensuring that users are authenticated and authorized to access specific resources. The resource manager 111 communicates with a data store 112 to retrieve account data and other relevant information necessary for managing user interactions with the system. The system is also operable to authenticate users through various means, including multi-factor authentication or biometric data, ensuring secure access to sensitive medical information.

In various embodiments, the host machine 121 can be operable to host the integration service 120. Multiple host machines can be instantiated to handle different users or clients, enabling the system to scale based on demand. The cloud-based nature of the system ensures that multiple users can access the service simultaneously, from various locations, without experiencing delays or interruptions in the data processing and presentation.

The interface layer 108 provides an API or other exposed interfaces that enable users to submit requests to the resource provider environment 106. These APIs can be used to manage the system, activate instances of the integration service 120, and interact with the system in real-time. The interface layer 108 can also manage the authentication of user requests and ensure that the system operates within defined access controls.

FIG. 1B illustrates the network architecture of a remote examination system in accordance with various embodiments. In an embodiment, the system may be comprised of user device(s) 130, a vision intake system 132 with a 3D eye data datastore 133, a machine learning system 134 with a diagnostic data datastore 135, an integration system 136, a records system interface 138, mixed reality device(s) 140, and a network 104, over which the various systems and devices communicate and interact.

The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met.

Vision intake system 132 is operable to capture and process 3D video data of a patient's eye during an examination. In various embodiments, vision intake system 132 includes hardware and software components that capture high-resolution, three-dimensional images of the eye, allowing for real-time examination of its anatomical structures. For example, vision intake system 132 may capture 3D video data representing different regions of the eye, such as the cornea, retina, and optic nerve, enabling comprehensive analysis. In certain embodiments, vision intake system 132 utilizes computer vision algorithms to automatically identify, track, and focus on intraocular structures during the exam, ensuring that the capture process is aligned with the doctor's field of interest.

In an embodiment, computer vision algorithms dynamically adjust the focus, lighting, and capture parameters of the 3D video feed based on the detected structures within the eye. For instance, when the system detects the pupil or other key anatomical features, it adjusts the focus and exposure settings to capture a clearer image of the surrounding structures. Additionally, the system is operable to apply segmentation models in real-time to delineate regions such as the cornea, iris, lens, and retina. This allows the system to provide continuous feedback to the operator, ensuring optimal capture of the targeted areas and minimizing noise or unnecessary data. In various embodiments, the system integrates with robotic components, such as a slit lamp mounted on servos, to dynamically adjust focus and centration throughout the examination or treatment process. The servos, guided by 3D computer vision algorithms, ensure proper alignment and focus, addressing challenges in maintaining steady visualization of the eye during both diagnostics and treatment.

Once the 3D video data is captured, it is transmitted to 3D eye data datastore 133, which is operable to store and manage this data. In certain embodiments, 3D eye data datastore 133 serves as a repository for storing the captured 3D video data, including stereoscopic data, depth maps, and other processed imaging formats. For example, the datastore may apply compression techniques to optimize storage capacity while maintaining the quality of the video data for future retrieval and analysis. In some embodiments, 3D eye data datastore 133 is configured to organize the captured data by the specific region of the eye under examination. For instance, it may categorize the data into datasets for different areas such as the cornea, retina, or optic nerve, ensuring that the appropriate data can be retrieved for real-time analysis or further processing by other components, such as machine learning system 134. In certain embodiments, 3D eye data datastore 133 may also store focus and alignment data generated by the servos and computer vision algorithms during the examination, allowing for review and refinement of treatment procedures based on historical data.

In another embodiment, computer vision algorithms within vision intake system 132 enable real-time analysis of focus and alignment during treatment, ensuring that optical equipment, such as robotic slit lamps, remain centered on the target areas of the eye. The system can automatically adjust focus during treatment, making corrections to ensure accurate targeting for procedures such as laser treatments or other interventions. The 3D video data captured during this process can also be used for post-treatment review, enabling doctors to monitor the precision of the treatment and identify any areas that may require further attention.

Alternatively, vision intake system 132 could use different types of cameras or sensors to capture the 3D video data. For example, the system might employ stereoscopic cameras, depth sensors, or time-of-flight cameras, depending on the specific requirements of the examination. In some cases, the system could also use multiple cameras positioned at different angles to create a more comprehensive 3D representation of the eye. The system could integrate these camera feeds into a unified visualization, allowing the doctor to view the eye from different perspectives in real time, with the help of automatic focus adjustments from the computer vision algorithms.

In an embodiment, vision intake system 132 applies pre-processing techniques, such as noise reduction and image stabilization, to enhance the quality of the 3D video data before it is stored in 3D eye data datastore 133. In one embodiment, if there is movement during the examination, the system can apply a motion-detection algorithm to stabilize the image, minimizing artifacts that might otherwise affect subsequent analysis. The system may also apply additional computer vision-based correction techniques to account for distortions or aberrations in the captured video feed, further improving the quality of the visual data.

In various embodiments, vision intake system 132 incorporates real-time calibration tools that work in conjunction with the computer vision algorithms. These calibration tools ensure that the captured 3D video data is aligned with the patient's anatomy. For example, the system may overlay reference points or markers on the video feed based on the detected structures, allowing for precise measurements and real-time adjustments. In certain embodiments, the calibration system is operable to adjust the field of view or zoom level dynamically as the system detects and tracks the regions of interest within the eye. For treatment guidance, the system may prompt the operator to refocus on specific areas of interest or make adjustments during procedures, such as laser-based therapies.

In various embodiments, vision intake system 132 and 3D eye data datastore 133 enable real-time data capture and processing, ensuring that the captured 3D video is available for immediate analysis. The system can continuously apply segmentation and tracking models to provide real-time feedback during the examination or treatment, ensuring that the captured data is relevant and focused on the necessary anatomical structures. This continuous data flow allows the system to provide up-to-date information during patient examinations and treatment, and ensures that the data is ready for integration with other components, such as machine learning system 134, for generating diagnostic insights or guiding treatment.

Machine learning system 134 is trained to detect potential abnormalities in the anatomical structures of the eye based on the 3D data captured by vision intake system 132. The system applies machine learning models, including convolutional neural networks (CNNs) and other computer vision techniques, to analyze the captured data and compare it against a dataset of annotated eye images and videos. The machine learning models are operable to identify patterns indicative of common ophthalmic conditions, such as glaucoma, macular degeneration, or retinal detachment.

For example, the system may analyze changes in retinal thickness or detect abnormal optic nerve cupping, which are indicative of early-stage glaucoma. The machine learning models are designed to flag these abnormalities in real-time, providing diagnostic insights that assist the healthcare professional in making informed decisions. The results of the analysis, including any identified abnormalities, are integrated into the 3D visualization and presented through the mixed reality interface, where the user can further investigate the flagged areas.

In some embodiments, machine learning system 134 continuously improves its accuracy by learning from new data captured during examinations, incorporating feedback from healthcare professionals to refine its diagnostic capabilities over time. As new diagnostic data and outcomes from patient exams are fed into the system, the machine learning models are updated to reflect more precise and accurate results, adapting to a variety of patient conditions and improving overall diagnostic performance.

In addition to detecting abnormalities, machine learning system 134 can also determine the current stage of an ophthalmic examination based on the anatomical structures identified in the 3D data. By comparing changes in the anatomical structure of the eye to predefined stage models, the system can determine whether the examination is focusing on the cornea, optic nerve, or other key regions of the eye, and adjust the diagnostic focus accordingly. The determination of the examination stage enables the system to retrieve and present relevant diagnostic data for the corresponding phase, enhancing the precision of the examination and providing contextually appropriate data to the healthcare professional.

Diagnostic data datastore 135 is operable to store diagnostic results generated by machine learning system 134. In an embodiment, diagnostic data may include condition-specific metrics, such as optic nerve thickness, retinal layer measurements, or corneal topography. For example, machine learning system 134 may analyze data from the retina to identify early signs of macular degeneration by comparing the current retinal thickness to a set of historical averages. Diagnostic data datastore 135 indexes and stores these results, allowing for retrieval by other components of the system, such as the mixed reality interface. In certain embodiments, machine learning system 134 continuously updates these metrics during live procedures, allowing the system to adjust the focus or alignment of a robotic slit lamp, laser system, or autofocus mechanism within the camera via a computer vision feedback loop in real-time to improve the accuracy of the treatment.

In various embodiments, machine learning system 134 interacts with vision acquisition enhancement component 213 and vision data analysis component 212 to process the segmented video data and apply diagnostic algorithms. For instance, machine learning system 134 may utilize deep learning models to analyze segmented data related to the cornea, assessing corneal thickness and identifying possible corneal dystrophies and pathologies. The results of these models, including both raw measurements and diagnostic classifications, can be stored in diagnostic data datastore 135 for further analysis or retrieval. In some cases, machine learning system 134 provides feedback to adjust the positioning of diagnostic or treatment devices, ensuring that the system remains properly aligned with the target structures within the eye.

In an embodiment, machine learning system 134 applies a combination of convolutional neural networks (CNNs) and decision trees to classify specific eye conditions based on the segmented data provided by vision data analysis component 212. For example, after vision data analysis component 212 isolates a segment of the optic nerve, machine learning system 134 can use a trained CNN to determine whether there are signs of optic neuropathy, such as an abnormally small optic nerve cup-to-disc ratio. The results of this classification are stored in diagnostic data datastore 135 for retrieval by a healthcare professional. Additionally, machine learning system 134 may guide treatment interventions, ensuring that the focal point of the slit lamp or other device remains centered on the correct ocular structure during a procedure.

In an embodiment, diagnostic data datastore 135 is operable to organize and store diagnostic data generated by machine learning system 134 in a format that facilitates rapid retrieval and display. For example, the datastore may organize diagnostic data based on specific patient identifiers, eye regions analyzed, and timestamps. This enables medical professionals to track diagnostic results over time or compare them with historical data, improving decision-making during follow-up examinations or treatments.

In certain embodiments, machine learning system 134 applies diagnostic models to segmented regions of the eye over time, detecting changes in structure or health. For instance, the system may track changes in the thickness of the retinal nerve fiber layer over multiple examinations and use this information to flag potential glaucoma progression. In some embodiments, diagnostic models are updated regularly based on new patient data, ensuring that the system continually refines its predictions and classifications. During treatment, machine learning system 134 may use these trends to adjust real-time focus and alignment, ensuring that treatments such as laser surgeries and UV light procedures are accurately targeted based on current and historical eye data.

Machine learning system 134 is operable to analyze data in both batch and continuous modes, depending on the clinical context. In continuous mode, machine learning system 134 can process new 3D video data as it becomes available, generating diagnostic results in near real-time. In batch mode, the system can analyze historical data sets stored in diagnostic data datastore 135, identifying long-term trends in patient eye health. In certain embodiments, machine learning system 134 updates its models based on new data, improving its diagnostic capabilities over time. During live procedures, the system can dynamically adjust focus and positioning, guiding robotic slit lamps and a camera lens, or lasers to ensure accurate alignment during treatment.

There are alternatives to the machine learning system 134. For example, a system could use traditional image processing techniques to analyze the 3D eye video data and other clinical information. This alternative system would rely on pre-defined rules and heuristics to identify specific eye regions and potential diagnostic information, rather than using machine learning algorithms. Another alternative could involve a system that uses a combination of machine learning and traditional image processing techniques. This hybrid system would apply both machine learning models and pre-defined rules to the data it receives, potentially offering a balance between the adaptability of machine learning and the reliability of traditional image processing techniques. In either case, the system may still assist in guiding treatment procedures by maintaining focus and alignment on the eye structures of interest.

Integration system 136 is operable to serve as a central hub for managing and coordinating the flow of data between various components of the remote ophthalmic examination system. In various embodiments, integration system 136 is responsible for receiving, processing, and transmitting data from multiple sources, including vision intake system 132, machine learning system 134, records system interface 138, and mixed reality device(s) 140. Integration system 136 is configured to ensure that the different data types, including 3D video data, diagnostic information, and patient records, are properly synchronized and formatted for use across the various system components.

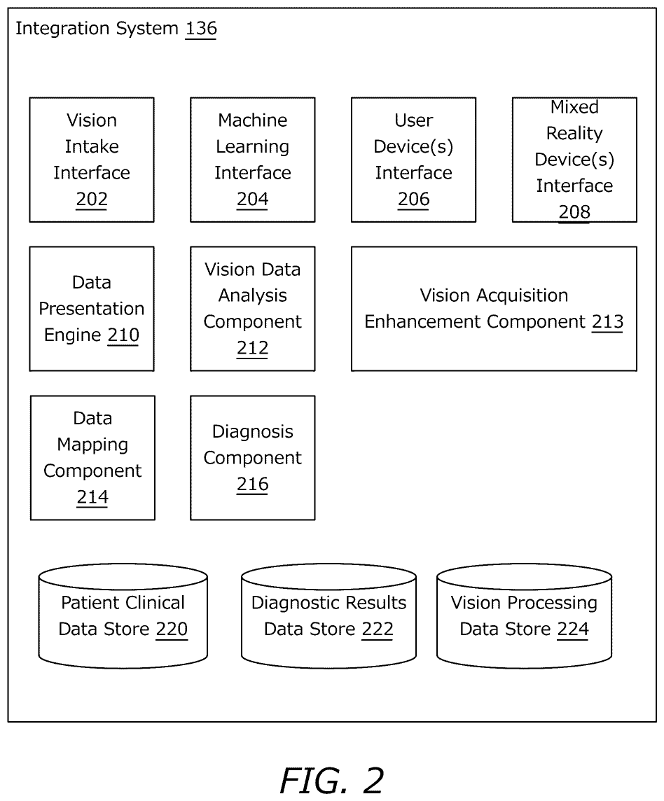

In an embodiment, integration system 136 includes various interfaces that allow for communication between the different subsystems. For example, integration system 136 can receive the 3D video data captured by vision intake system 132 and the diagnostic results generated by machine learning system 134. As will be described further in FIG. 2, these components are managed and routed through the integration system to ensure that relevant data is provided to the appropriate system, such as presenting the diagnostic information on mixed reality device(s) 140.

Integration system 136 is operable to apply data processing techniques to ensure compatibility and coherence between various data formats. For example, integration system 136 can normalize data from disparate sources, converting the 3D video data from vision intake system 132 and diagnostic results from machine learning system 134 into formats that can be interpreted and displayed by mixed reality device(s) 140. In some embodiments, integration system 136 coordinates the retrieval of patient records from records system interface 138, ensuring that the retrieved records are properly aligned with the real-time diagnostic results generated by the machine learning system.

In various embodiments, integration system 136 includes a data presentation engine, which is operable to format and prepare the data for display on mixed reality device(s) 140. For example, the data presentation engine can integrate the 3D video feed with diagnostic information and patient records, ensuring that all necessary data is synchronized and presented cohesively to the healthcare professional. As will be described further in FIG. 2, the data presentation engine formats the processed data for mixed reality display.

In an embodiment, integration system 136 is responsible for managing the communication between the various components over network 104. The system ensures that data flows securely and efficiently across the network, enabling real-time interactions between the different subsystems. For example, integration system 136 may receive 3D video data and diagnostic results, process the data, and then transmit it to mixed reality device(s) 140, enabling the healthcare provider to access the integrated information during the examination.

Alternatively, the integration system 136 could be designed to handle data flow in a different manner. For example, instead of receiving data directly from each component, the integration system 136 could access a centralized database where all the data is stored. Another alternative could involve using a distributed architecture, where each component of the remote examination system has its own dedicated data processing module. In this case, the integration system 136 would be responsible for coordinating the flow of data between these modules.

The integration system 136 could employ various techniques to optimize the data processing and transmission process. For instance, it could use data compression algorithms to reduce the amount of data being sent to the mixed reality interface, or it could implement caching mechanisms to store frequently accessed data locally, reducing the need for repeated data retrieval from the datastores.

In various embodiments, integration system 136 includes logging and monitoring functionalities that track the data exchanged between the components. This system is operable to log data access, modifications, and transmissions, ensuring the integrity and traceability of the data being used across the system.

Records system interface 138 is operable to facilitate communication between the remote ophthalmic examination system and external patient records databases or electronic health record (EHR) systems. In various embodiments, records system interface 138 is responsible for retrieving, updating, and storing patient records, including historical medical data, examination results, and treatment plans. The interface ensures that patient-specific data is available for integration with real-time diagnostic results and 3D video data during the examination.

In an embodiment, records system interface 138 is operable to query external databases, such as hospital EHR systems or specialized ophthalmic databases, to retrieve relevant patient information. For example, records system interface 138 can retrieve past examination results, such as optical coherence tomography (OCT) scans, intraocular pressure (IOP) measurements, and previous diagnoses, and make this data available for use by other system components, such as integration system 136 or machine learning system 134.

In various embodiments, records system interface 138 is responsible analyzes and process patient data to format the data to be compatible with other data formats used in the system. For instance, the interface may convert the retrieved medical records into a format that can be processed by integration system 136 or displayed on mixed reality device(s) 140. As will be described further in FIG. 2, the interface ensures that patient records are synchronized with real-time diagnostic information for seamless integration during an examination.

In certain embodiments, records system interface 138 is operable to update patient records based on new examination results or treatment decisions. For example, after the machine learning system 134 generates diagnostic results, records system interface 138 may store these new results in the external patient records database, ensuring that the patient's medical history is up-to-date and accessible for future use.

In various embodiments, records system interface 138 is responsible for managing secure access to patient records. The interface may include encryption protocols and authentication mechanisms to ensure that only authorized personnel can access or modify patient data.

In an embodiment, records system interface 138 works in conjunction with integration system 136 to synchronize retrieved patient data with the 3D video data captured by vision intake system 132 and the diagnostic results generated by machine learning system 134. For example, during an examination, the system may retrieve historical OCT scans from the patient's records, and records system interface 138 ensures that this data is properly integrated and available for comparison with real-time diagnostic results.

Mixed reality device(s) 140 are operable to display 3D video data, diagnostic information, and treatment guidance to medical professionals in an immersive environment during remote ophthalmic examinations. In various embodiments, mixed reality device(s) 140 include hardware and software components that enable the user to interact with a combination of real-world and digital content, such as 3D video feeds of the patient's eye and related diagnostic information, within a virtual or augmented reality interface. Mixed reality device(s) 140 also aid in treatment processes by displaying real-time feedback and adjustments, driven by computer vision algorithms for enhanced focus and precision during ophthalmic procedures.

In an embodiment, mixed reality device(s) 140 are operable to receive 3D video data captured by vision intake system 132 and formatted by integration system 136. The device can present this data to the healthcare professional as a fully immersive visualization of the patient's eye, allowing the user to examine the anatomical structures in detail. The system can overlay diagnostic data generated by machine learning system 134, such as measurements of retinal thickness or optic nerve analysis, corneal tomography, directly onto the 3D visualization, providing a combined view of the video data, diagnostic information, and treatment recommendations. For instance, the system may dynamically adjust the display based on real-time computer vision analysis, suggesting optimal regions of focus for treatment or highlighting anatomical structures where action is required.

In various embodiments, mixed reality device(s) 140 are capable of interacting with the healthcare professional's gestures, voice commands, or other input methods to adjust display and interaction parameters. For instance, the healthcare professional can zoom in on specific regions of the 3D video or highlight relevant diagnostic data for closer examination. The system supports a range of inputs, such as hand gestures or voice prompts, to provide an intuitive, hands-free user experience during the examination. In some embodiments, the system may automatically guide the focus to critical areas of the eye based on computer vision analysis, ensuring that the most relevant structures are emphasized during treatment.

In an embodiment, mixed reality device(s) 140 can include virtual reality (VR) headsets, augmented reality (AR) glasses, or other appropriate wearable devices. These devices are operable to render both stereoscopic and monocular 3D video data, allowing the user to visualize the eye's anatomy with depth perception. For example, a VR headset may provide the healthcare professional with a fully immersive experience, displaying the 3D video data from the examination in real-time alongside relevant diagnostic information, patient records, and live treatment guidance. Additionally, the device may support automatic adjustments to the field of view or zoom level, optimizing visualization based on detected structures within the eye.

In various embodiments, mixed reality device(s) 140 are operable to receive and display information processed by data presentation engine 210 of integration system 136. This engine is responsible for formatting the 3D video data, diagnostic information, treatment feedback, and patient records for display on mixed reality device(s) 140. As a result, the device is capable of presenting a synchronized and cohesive view of the patient's eye examination, including any relevant diagnostic results, historical patient data, and live treatment updates. The system may also suggest actions based on real-time analysis, such as centering the view on regions requiring medical intervention or guiding the focus for a precise laser treatment.

For example, mixed reality device(s) 140 are operable to present an interactive visualization of the 3D eye data, which includes user-controllable elements for manipulating diagnostic overlays. The system enables the user, such as a healthcare professional, to interact with the overlaid diagnostic information using various input methods, including hand gestures, voice commands, or direct selection via touchscreen interfaces. For instance, during a remote ophthalmic examination, the user may perform a hand gesture to zoom in on a specific region of the 3D eye model, such as the retina, and view more detailed diagnostic information, such as retinal thickness measurements.

In certain embodiments, the system allows the user to toggle diagnostic overlays, enabling or disabling specific layers of information. For example, the user may choose to focus on a particular diagnostic parameter, such as intraocular pressure, and hide other irrelevant metrics during the examination. The user can also reposition diagnostic elements within the field of view, adjusting the layout of the mixed reality display to suit their workflow. These interactions are supported by a gesture-based input interface, which tracks hand movements in real-time and translates them into corresponding system commands.

Additionally, the system is operable to allow users to annotate the 3D eye model during the examination. For instance, the user may draw on the model to highlight areas of interest, adding notes or tagging regions that require further investigation. These annotations are saved alongside the diagnostic data and can be referenced during follow-up examinations or for medical record-keeping purposes.

User device(s) 130 can include, generally, any computing device that is operable to communicate over a network 104. Data may be collected from user device(s) 130, and data requests may be initiated by each user device 130. The user device(s) 130 may be a server, a desktop computer, a laptop computer, a tablet, a smartphone, or any other suitable computing device. User device(s) 130 may execute one or more applications, such as a web browser or a dedicated application that can facilitate interactions with the remote examination system.

In certain embodiments, mixed reality device(s) 140 may include tools for the healthcare professional to interact with the diagnostic data and treatment guidance in real-time. For example, the device may allow the user to annotate areas of the 3D video, add notes, or highlight regions of interest for further investigation or treatment. This annotated data may be stored in the system or sent back to the diagnostic data datastore 135 for future reference or additional analysis. The system may also provide visual cues, such as color-coded regions, to indicate areas requiring further inspection or treatment during the procedure.

In an embodiment, mixed reality device(s) 140 are operable to work in conjunction with integration system 136 to update the display in real-time as new data is captured or processed. For example, as machine learning system 134 generates new diagnostic insights or treatment recommendations, mixed reality device(s) 140 can dynamically update the display to reflect these changes, providing the healthcare professional with up-to-date information throughout the examination. The system may also provide live feedback during treatment, such as adjusting focus or suggesting real-time corrections based on computer vision analysis.

In various embodiments, mixed reality device(s) 140 may include features to assist with treatment precision, such as visual prompts or overlays that guide the user to maintain optimal focus and positioning during laser treatments, ultraviolet light treatments, or other procedures. These prompts can be driven by real-time computer vision algorithms that detect suboptimal conditions and suggest corrective actions, ensuring accurate and effective treatment delivery.

User device(s) 130 can be an electronic device including hardware, software, or embedded logic components, or a combination of two or more such components, capable of carrying out the appropriate functions implemented or supported by the system. For example, user device 130 may include a mobile phone, tablet computer, or laptop, enabling users, such as medical professionals, to interact with the system remotely. User device 130 may also enable network access to the system and facilitate real-time communication with other components, such as vision intake system 132 or machine learning system 134.

User device(s) 130 can include web browsers, such as MICROSOFT INTERNET EXPLORER, GOOGLE CHROME, GOOGLE MEET, or MOZILLA FIREFOX, that allow users to enter Uniform Resource Locators (URLs) directing the browser to a server, which generates and communicates Hyper Text Transfer Protocol (HTTP) requests. Servers may accept these HTTP requests and respond with appropriate files, such as Hyper Text Markup Language (HTML) files, which user device(s) 130 can render as web pages for the user. In various embodiments, the user device 130 may also run dedicated applications loaded onto the device that are operable to obtain and display data from network 104 within an application interface.

Exemplary user devices include desktop computers, laptops, tablets, smartphones, and other mobile devices that are operable to perform the functions described herein. The present disclosure contemplates any suitable user device(s), including those capable of accessing network 104 and interacting with the system in real time, through cloud-based or locally installed applications. Where appropriate, user device(s) 130 may enable medical professionals to remotely access examination data and interact with other system components to facilitate diagnosis and patient care.

Network 104 generally represents a network or collection of networks (such as the Internet or a corporate intranet, or a combination of both) over which the various components illustrated in FIG. 1B (including other components that may be necessary to execute the system described herein, as would be readily understood to a person of ordinary skill in the art). In particular embodiments, network 104 is an intranet, an extranet, a virtual private network (VPN), a local area network (LAN), a wireless LAN (WLAN), a wide area network (WAN), a metropolitan area network (MAN), a portion of the Internet, or another network 104 or a combination of two or more such networks. One or more links connect the systems and databases described herein to the network 104. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network, and any suitable link for connecting the various systems and databases described herein.

The network 104 connects the various systems and computing devices described or referenced herein. In particular embodiments, network 104 is an intranet, an extranet, a virtual private network (VPN), a local area network (LAN), a wireless LAN (WLAN), a wide area network (WAN), a metropolitan area network (MAN), a portion of the Internet, or another network or a combination of two or more such networks. The present disclosure contemplates any suitable network.

One or more links couple one or more systems, engines or devices to the network 104. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 104.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiment, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1B, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

FIG. 2 illustrates an example computing environment including an integration system in accordance with an exemplary embodiment. In this example, integration system 136 includes vision intake interface 202, machine learning interface 204, user device(s) interface 206, mixed reality device(s) interface 208, data presentation engine 210, vision data analysis component 212, vision acquisition enhancement component 213, data mapping component 214, diagnosis component 216, patient clinical data store 220, diagnostic results data store 222, and vision processing data store 224.

Vision intake interface 202 is operable to manage the communication and data flow between vision intake system 132 and other components within integration system 136. More specifically, vision intake interface 202 receives the 3D video data captured by vision intake system 132 and ensures that the data is properly processed and formatted for use by other system components, such as vision acquisition enhancement component 213 and vision data analysis component 212.

For example, in an embodiment, vision intake interface 202 is operable to preprocess the incoming 3D video data from vision intake system 132. This preprocessing may include data formatting, noise reduction, and applying compression techniques to ensure efficient storage and transmission. For instance, vision intake interface 202 may employ compression techniques, such as lossless or lossy compression, to optimize the size of the video data while maintaining the quality necessary for analysis. Once processed, the data may be transmitted to vision processing data store 224 for storage or passed on for further analysis by system components.

In various embodiments, vision intake interface 202 is operable to transmit the preprocessed 3D video data to vision acquisition enhancement component 213 for additional adjustments, such as focus refinement and image stabilization. Following these adjustments, the video data may be processed by vision data analysis component 212 to segment and identify specific anatomical structures of the eye.

In certain embodiments, vision intake interface 202 is responsible for ensuring that the data flow between vision intake system 132 and data presentation engine 210 remains synchronized. For example, vision intake interface 202 may format the 3D video data for smooth integration with diagnostic outputs generated by machine learning system 134 or contextual information, such as historical medical records retrieved from patient clinical data store 220. This ensures that the information is aligned for display on mixed reality device(s) 140.

In some embodiments, vision intake interface 202 may operate using various communication protocols to facilitate efficient data transmission. For instance, vision intake interface 202 may use protocols such as REST API, WebSocket, or other real-time communication protocols like Real-Time Transport Protocol (RTP) or Real-Time Streaming Protocol (RTSP) for transmitting high-speed, low-latency 3D video data. In alternative configurations, vision intake interface 202 could employ a message queue or publish-subscribe system, allowing the 3D video data to be asynchronously transferred between vision intake system 132 and integration system 136. This may decouple these systems, improving scalability and reliability.

In various embodiments, vision intake interface 202 is responsible for ensuring the integrity and accuracy of the data it processes. The interface may apply checksums, error correction algorithms, or other validation techniques to ensure that the data transmitted from vision intake system 132 remains uncorrupted. This ensures that all subsequent diagnostic analysis and presentation rely on accurate, unaltered data.

Machine learning interface 204 is operable to manage the communication and data exchange between machine learning system 134 and other components within integration system 136. More specifically, machine learning interface 204 transmits data, such as 3D video segments, diagnostic outputs, and contextual information, to and from machine learning system 134. This interface facilitates the flow of information necessary for the system's diagnostic processes, ensuring that the machine learning models have access to the relevant input data and that the results are available for further processing and display.

For example, in an embodiment, machine learning interface 204 is operable to receive segmented 3D video data from vision data analysis component 212 and transmit this data to machine learning system 134 for analysis. Machine learning system 134 may apply algorithms, such as convolutional neural networks (CNNs), to identify specific anatomical structures of the eye, including retinal layers, corneal thickness, lens/cataract grade, or optic nerve contours. The diagnostic results produced by these models are then transmitted back through machine learning interface 204 for further processing, display, or storage.

In various embodiments, machine learning interface 204 is operable to transmit diagnostic data generated by machine learning system 134 to diagnostic data datastore 135. The transmitted data may include classification outputs, anomaly detections, or predictive insights generated by the machine learning models. For instance, if machine learning system 134 identifies patterns consistent with macular degeneration, this diagnostic output can be stored in diagnostic data datastore 135 for further review or integration with the patient's medical history.

In certain embodiments, machine learning interface 204 supports interactions between machine learning system 134 and other components, such as vision acquisition enhancement component 213. This allows the machine learning models to provide feedback to optimize the image capture process. For instance, machine learning system 134 may detect blurriness or focus issues in the 3D video data and adjust the capture settings through communication with vision intake system 132.

[In an embodiment, machine learning interface 204 is responsible for managing the data flow necessary for training and refining the machine learning models. This may involve retrieving historical diagnostic data and patient records from diagnostic data datastore 135 or other data sources. For example, training data may include prior OCT scans, fundus images, intraocular pressure measurements, corneal tomography, topography, or ocular biometry, which are fed into machine learning system 134 to improve its diagnostic accuracy. Once the models are updated, machine learning interface 204 routes the updated models back into the system for use during patient examinations.

In various embodiments, machine learning interface 204 is operable to integrate external machine learning models or algorithms into the system. For example, pre-trained models from external sources may be incorporated into machine learning system 134 for additional diagnostic capabilities, such as detecting glaucoma or cataracts. Machine learning interface 204 facilitates the ingestion of these models and their application to the 3D video data captured by vision intake system 132.

In certain embodiments, machine learning interface 204 ensures the integrity of the data exchanged between machine learning system 134 and other components by applying data validation techniques, such as checksums or error-correcting codes. This ensures that the transmitted data, including sensitive diagnostic outputs and 3D video segments, remains accurate and intact during processing and storage.

In another embodiment, machine learning interface 204 may operate using various communication protocols for transmitting data between machine learning system 134 and other components. For example, machine learning interface 204 could employ REST API, WebSocket, or other real-time communication protocols to transmit data over the network efficiently. In some cases, a message queue or publish-subscribe system may be used to manage the asynchronous transfer of data, enabling more scalable and flexible communication between components User device(s) interface 206 is operable to manage the communication and data flow between user device(s) 130 and integration system 136. More specifically, user device(s) interface 206 facilitates the exchange of commands, requests, and display data between user device(s) 130 and the other components of integration system 136, allowing users, such as doctors or technicians, to interact with the system during patient examinations.

In various embodiments, user device(s) interface 206 is responsible for transmitting diagnostic outputs, 3D video segments, and contextual information to user device(s) 130 for display and interaction. For example, user device(s) interface 206 may transmit segmented 3D video data from vision intake system 132 to a tablet or workstation used by the ophthalmologist, enabling real-time visualization and manual adjustments during an examination. The interface may also receive commands from user device(s) 130, such as selecting specific exam data, adjusting video playback, or requesting diagnostic results.

For example, in an embodiment, user device(s) interface 206 is operable to handle input from various user devices, including desktop computers, mobile devices, or wearable devices, such as smart glasses. The interface is designed to accommodate different device types by supporting multiple communication protocols, such as HTTP, WebSocket, or Bluetooth, to enable seamless integration across different environments. For instance, a doctor may use a tablet to zoom in on specific regions of the 3D video or issue commands to retrieve patient history or diagnostic information in real-time.

In certain embodiments, user device(s) interface 206 is operable to manage various data types received from user device(s) 130, such as manual inputs, voice commands, or gesture-based interactions. For example, a doctor may use hand gestures to zoom in on a specific part of the 3D video feed, or a voice command may be issued to retrieve relevant diagnostic data from diagnostic data datastore 135 for display. User device(s) interface 206 ensures that these inputs are processed correctly and transmitted to the appropriate components within integration system 136.

In an embodiment, user device(s) interface 206 may support encrypted communication protocols, such as TLS/SSL, to secure the data transmitted between user device(s) 130 and the system. This ensures that sensitive patient data remains protected during transmission. Additionally, the interface may apply compression algorithms to optimize the transmission of large datasets, such as 3D video segments, without compromising the quality of the data displayed on user device(s) 130.

In various embodiments, user device(s) interface 206 is operable to synchronize the data displayed on user device(s) 130 with the data being processed by other system components. For example, real-time updates from machine learning system 134, such as diagnostic results or segmentation data, can be transmitted through user device(s) interface 206 to ensure that the user always has access to the latest information.

User device(s) interface 206 is operable to handle multiple user sessions concurrently. This may be achieved through session management protocols that track the inputs and outputs for each user device separately, allowing multiple doctors or technicians to interact with the system simultaneously, each receiving personalized data streams relevant to their specific tasks or patients.

Alternatives to user device(s) interface 206 may include a direct connection between user device(s) 130 and integration system 136, bypassing the need for a dedicated interface. However, this direct connection may complicate the communication process by introducing tighter coupling between components. Another alternative could involve the use of a multi-interface architecture, where several distinct interfaces manage the communication between different parts of the system. This setup would increase system flexibility, allowing different user devices to interact with specific components independently, but at the cost of increased complexity in data routing and session management.

In various embodiments, user device(s) interface 206 ensures the integrity of the data exchanged by applying data validation techniques, such as checksums or error-detection codes. This ensures that the data transmitted between user device(s) 130 and the system remains accurate and unaltered during the transfer process.

In one embodiment, mixed reality device(s) interface 208 serves as a communication bridge between mixed reality device(s) 140 and integration system 136. At a high level, mixed reality device(s) interface 208 is responsible for transmitting 3D video data and associated contextual information from integration system 136 to mixed reality device(s) 140, enabling the data to be displayed in a virtual or augmented reality environment.

Mixed reality device(s) interface 208 operates by establishing a continuous connection with mixed reality device(s) 140 and transmitting processed examination data, such as 3D eye video and diagnostic results, received from integration system 136. As the data is transmitted, mixed reality device(s) interface 208 ensures that it is properly formatted, optimized for display, and synchronized across devices. This may involve applying compression techniques, such as H.264 or HEVC, to minimize bandwidth requirements or using specialized communication protocols to reduce latency and ensure smooth video playback in the mixed reality environment.

Once the data has been transmitted, mixed reality device(s) 140 overlays the information in a virtual or augmented reality environment, allowing medical professionals to visualize the 3D eye data alongside diagnostic information in real-time. The system continuously updates the displayed information as new data is processed by integration system 136, ensuring that the user has access to the most current and relevant information at all times. For example, diagnostic results generated by machine learning system 134 can be displayed as annotations or overlays on the 3D video, highlighting areas of concern within the patient's eye, such as retinal abnormalities, optic nerve damage, or anterior chamber pathology.

In certain embodiments, mixed reality device(s) interface 208 is operable to manage simultaneous connections with multiple mixed reality devices. For example, multiple doctors or technicians in different locations could access the system at the same time, each receiving their own tailored data stream. One user may be conducting a live eye examination while another reviews past diagnostic results, with all interactions synchronized in real-time across devices.

Alternatives to mixed reality device(s) interface 208 could involve different communication protocols for transmitting data. For example, the interface could use a wireless protocol, such as Wi-Fi, Bluetooth, or 5G, to allow for more mobility during an examination, enabling the doctor to move freely without being tethered to a stationary system. Alternatively, a wired connection, such as HDMI or USB-C, may be used to ensure a more stable connection, reducing the risk of signal loss or interference, particularly in environments where wireless connections may be unstable.

In various embodiments, mixed reality device(s) interface 208 is operable to process interactive commands from the user, such as hand gestures or voice commands. For example, a doctor wearing augmented reality glasses may use a hand gesture to zoom in on a specific region of the eye or employ a voice command to retrieve historical data from patient clinical data store 220. These commands are transmitted via mixed reality device(s) interface 208 to integration system 136, where they are processed, and the corresponding actions or data are relayed back to the mixed reality display in real-time.

In terms of data formatting and optimization, mixed reality device(s) interface 208 may use various formats depending on the capabilities of mixed reality device(s) 140. For example, the interface may encode video using standardized formats like MPEG-4 or H.264, or it may employ more advanced compression algorithms, such as H.265 or AV1, to reduce the bandwidth required for transmission without compromising the video quality. Additionally, the interface may optimize the data stream for device-specific features, adjusting resolution or frame rates depending on the hardware capabilities of the individual mixed reality device.

Furthermore, mixed reality device(s) interface 208 may apply error-checking algorithms, such as checksums or forward error correction (FEC), to ensure data integrity during transmission. This helps maintain the quality and accuracy of the transmitted 3D video and diagnostic information, particularly when wireless protocols are used, ensuring that even in environments with variable network performance, the data remains intact and suitable for medical diagnosis.

Data presentation engine 210 is operable to manage the integration and formatting of 3D video data, contextual information, diagnostic results, and treatment guidance for display within a mixed reality environment. More specifically, data presentation engine 210 assembles the data streams from various sources within integration system 136 and presents them in a coherent, user-friendly format on mixed reality device(s) 140, supporting both diagnosis and treatment processes. In certain embodiments, data presentation engine 210 is operable to display visual prompts or treatment guidance based on computer vision algorithms and diagnostic results, assisting medical professionals in making treatment decisions.

For example, data presentation engine 210 receives inputs from multiple system components, including 3D video data from vision intake system 132, contextual information from patient clinical data store 220, and diagnostic outputs from machine learning system 134. Data presentation engine 210 processes and integrates this data to ensure that the appropriate information is displayed alongside the relevant 3D video feed. For instance, while a doctor examines a patient's retina in 3D video, data presentation engine 210 can overlay diagnostic measurements, such as retinal thickness or intraocular pressure, within the mixed reality display. The system can also prompt the doctor to focus on specific regions of the eye based on pre-detected abnormalities or conditions requiring attention, such as directing focus to the optic nerve if machine learning system 134 flags potential glaucomatous damage.

In certain embodiments, data presentation engine 210 applies data synchronization techniques to ensure that the information presented in the mixed reality environment is consistent and up-to-date. This may involve time-stamping incoming data from various sources and using those timestamps to synchronize the 3D video with diagnostic and treatment information. For instance, if the diagnostic models detect changes in the patient's eye health, such as a deterioration in the optic nerve, data presentation engine 210 can ensure that these findings are aligned with the specific point in the 3D video where the abnormality is detected. The system may also overlay other imaging modalities such as OCT or visual field exams to allow for treatment guidance, such as recommended medication on interventional adjustments, based on real-time feedback from machine learning system 134.

In various embodiments, the system integrates the 3D video data with diagnostic data and patient-specific clinical information by mapping the relevant information to identified anatomical structures within the 3D video. For instance, diagnostic metrics such as intraocular pressure or corneal thickness can be mapped to the specific regions of the eye, such as the optic nerve or cornea, respectively.

In an embodiment, tagging techniques can be employed to associate key data points with the anatomical regions displayed in the 3D video. For example, if the system identifies the optic nerve during an examination, the system may automatically tag the optic nerve with associated diagnostic measurements and patient history, such as previous optic nerve scans or metrics that are relevant for comparison.

When a user interacts with a specific region of the 3D video, such as zooming in on the retina, the system dynamically links the region of interest to the corresponding diagnostic data and patient information. This linking enables real-time retrieval and display of relevant information, ensuring that the user has immediate access to the clinical context needed to assess the condition of the patient's eye.

In alternative embodiments, the system may use pre-defined mapping schemas or machine learning-based techniques to enhance the accuracy of the mapping process, adapting to different anatomical structures based on the specifics of each patient's eye and historical data.

Data presentation engine 210 is operable to optimize the format of the data for display on mixed reality device(s) 140. In certain embodiments, this involves converting the incoming data into compatible formats, such as converting diagnostic results into overlays or annotations that can be easily interpreted by the medical professional. For example, the engine may convert complex diagnostic outputs, such as a machine learning model's classification of potential pathologies, into visual cues like colored regions or textual annotations overlaid on the 3D video. Colors or graphical overlays may be used to emphasize specific areas that require closer examination, while additional layers may be shown or hidden depending on the doctor's preference, such as displaying only the corneal layer for analysis while suppressing views of the retina.

In various embodiments, the data presentation engine 210 is operable to map and tag diagnostic data to specific anatomical regions of the eye identified in the 3D data. As the system analyzes the captured 3D data and identifies key anatomical structures, such as the cornea, retina, lens, or optic nerve, the data presentation engine automatically associates relevant diagnostic data, patient-specific clinical information, and historical records with the corresponding regions of the 3D eye model. This mapping process is performed in real-time, ensuring that any updates to the anatomical data are immediately reflected in the diagnostic overlays.

In some embodiments, the mapping is achieved using landmark detection algorithms that identify specific reference points or regions on the 3D eye model. For example, the system may identify key anatomical landmarks, such as the optic disc or macula, and use these points as reference areas to which diagnostic measurements, such as retinal thickness or intraocular pressure, can be mapped. Tagging techniques are applied to associate each diagnostic metric with its corresponding anatomical region, allowing the user to interact with the 3D model and retrieve detailed information about specific areas of interest.

Additionally, the system may store these mappings in a structured format, such as a database of tagged regions, which can be referenced during future examinations to track changes over time. In certain embodiments, the system is operable to apply color-coded overlays, highlighting regions that require further attention based on the mapped diagnostic data.

In various embodiments, data presentation engine 210 uses different rendering techniques to present the data in the mixed reality environment. These rendering techniques may include volumetric rendering for 3D video data, where the anatomical structures of the eye are displayed in three dimensions, and 2D overlays for diagnostic or contextual information. For example, the system may display a 3D model of the retina while simultaneously presenting 2D graphs representing changes in intraocular pressure over time. Additionally, data presentation engine 210 can prompt the user to review specific sections of the eye, automatically guiding focus to critical areas during procedures.

Data presentation engine 210 optimizes the data for different types of mixed reality devices. This may include adjusting the data resolution, frame rate, or compression settings based on the device's capabilities. For example, if mixed reality device(s) 140 is a high-resolution VR headset, data presentation engine 210 may format the 3D video data in a high-resolution format to ensure detailed visualization. Alternatively, for lower-spec devices, the engine may apply lossy compression techniques to reduce the data size while maintaining sufficient visual clarity for the diagnosis and treatment.

In an embodiment, data presentation engine 210 supports interactive features that allow users to manipulate the displayed data. Medical professionals using mixed reality device(s) 140 may use hand gestures, voice commands, or controllers to interact with the presented data. Data presentation engine 210 processes these interactions and dynamically updates the display. For example, a doctor may zoom in on a specific region of the eye or toggle between different data layers, such as switching from a 3D view of the cornea to diagnostic overlays showing retinal health. The system may also allow the doctor to define or outline regions of the eye using gestures, prompting further analysis of those regions.

Data presentation engine 210 continuously updates the display as new diagnostic data or 3D video segments are processed by the system, ensuring that the doctor has access to the most current information throughout the examination. For example, if machine learning system 134 detects new abnormalities during the eye exam, the updated results are immediately integrated into the mixed reality view. In certain embodiments, this update process includes suggestions for real-time adjustments to ongoing treatment, such as refining laser focus during retinal treatments based on detected changes.

In various embodiments, data presentation engine 210 can be customized to display specific types of data based on user preferences or examination requirements. For instance, the doctor may choose to focus on certain diagnostic metrics, such as intraocular pressure or corneal thickness, and data presentation engine 210 will prioritize displaying this information. This customizable interface ensures that the most relevant information is always available to the doctor, and the system can prioritize treatment guidance based on the analysis of visual data.

There are alternative implementations of data presentation engine 210 depending on system requirements or performance needs. One alternative involves the use of edge computing, where data presentation engine 210 offloads some data processing tasks to local devices (e.g., mixed reality device(s) 140), reducing the burden on the central server and improving response times. In certain high-latency environments, handling data rendering directly on mixed reality device(s) 140 may improve real-time performance, while data presentation engine 210 manages data flow and synchronization. This can also include offloading treatment guidance calculations to the local device for faster decision-making.

In another embodiment, data presentation engine 210 could support the integration of external data sources, such as external medical databases or third-party diagnostic tools, incorporating additional medical data (e.g., pharmaceutical or genetic data) into the mixed reality environment. Data presentation engine 210 would ensure this data is formatted and synchronized with the patient's 3D eye video, diagnostic information, and real-time treatment feedback.

Additionally, data presentation engine 210 may implement machine learning models to automatically prioritize certain data for display. For example, the system could learn from a doctor's past preferences, automatically highlighting diagnostic information that the doctor frequently uses during an examination, and dynamically adjusting the display to offer treatment suggestions based on detected patterns.

In various embodiments, data presentation engine 210 supports augmented data layers, providing additional contextual information, predictive analytics, and treatment recommendations. These augmented data layers may present optional overlays that the doctor can enable or disable based on need, such as historical patient data, trend-based predictions of future eye health outcomes, or even suggested adjustments to treatment protocols.

Data presentation engine 210 also applies encryption techniques to ensure secure transmission of sensitive patient data, particularly when displayed on mixed reality device(s) 140 in a networked environment. The engine incorporates data validation protocols, such as checksums or secure hashing algorithms, to ensure diagnostic and contextual information remains accurate and uncorrupted during transmission and display, including critical treatment guidance information.

Vision data analysis component 212 is operable to process and analyze the 3D video data captured by vision intake system 132, providing assessments of the anatomical structures of the eye. In various embodiments, vision data analysis component 212 applies image processing algorithms to segment, identify, and classify different regions of the eye, such as the cornea, retina, and optic nerve, based on the incoming 3D video data. In some cases, computer vision techniques are utilized not only for diagnostics but also to provide feedback for treatment procedures, such as laser-based surgeries, by ensuring the system maintains proper alignment and focus on the relevant eye structures.

For example, vision data analysis component 212 can apply segmentation techniques to isolate specific layers of the retina or cornea for further examination. The segmentation process may involve the use of algorithms such as convolutional neural networks (CNNs) or active contour models, which are trained to detect the boundaries of anatomical structures with high precision. Once the segmentation is complete, vision data analysis component 212 is operable to apply further analysis to extract meaningful diagnostic data, such as measuring retinal thickness, detecting corneal abnormalities, or identifying optic nerve contours. In certain embodiments, vision data analysis component 212 works alongside servos and automated devices such as robotic slit lamps, which adjust focus and positioning during treatment based on real-time analysis of the 3D video data. In various embodiments, vision data analysis component 212, in conjunction with machine learning system 134, implements a computer vision feedback loop to perform autofocus adjustments during live procedures. The system processes real-time 3D video data captured by vision intake system 132 and continuously analyzes the visual data to detect shifts in the anatomical structures or changes in the focus of the imaging device. Based on the analysis, the system can dynamically adjust the camera's focus, ensuring that the target anatomical structures remain sharp and centered throughout the procedure. This autofocus capability is driven by the computer vision algorithms, which detect out-of-focus regions and adjust the lens automatically without requiring manual intervention, improving precision in critical procedures like laser surgeries and other corrective treatments. In various embodiments, the system is operable to determine a stage of an ophthalmic examination based on the anatomical structures identified in the captured 3D data. The system employs a combination of computer vision algorithms and machine learning models to analyze the progression of the examination. As the system captures 3D data of the patient's eye, the vision data analysis component 212 and machine learning system 134 are operable to detect specific anatomical features, such as the cornea, retina, iris, lens, or optic nerve. Based on changes in these structures over time, the system can infer the current phase of the examination.

For example, in a routine ophthalmic examination, different anatomical structures are observed in a predetermined sequence, such as an initial focus on the cornea followed by a deeper examination of the optic nerve. The system utilizes pre-defined models of these phases, including a correlation between the anatomical regions identified in the 3D data and the expected sequence of examination steps. In some embodiments, the system applies convolutional neural networks (CNNs) or similar machine learning techniques to continuously monitor changes in the eye's anatomical structures, comparing these changes to stored models of exam stages to determine the current stage of the examination.

In certain embodiments, the system is operable to present diagnostic data and recommendations specific to the current stage. For example, during the optic nerve evaluation phase, the system may automatically retrieve relevant diagnostic information, such as intraocular pressure data or historical optic nerve scans, to aid in real-time decision-making.

Alternatively, manual input from the medical professional may confirm or adjust the stage of the exam, allowing for a combination of automated and manual control. The system is further operable to retrieve diagnostic data and patient-specific clinical information associated with each stage, ensuring that the user has access to the relevant data for real-time decision-making.

In certain embodiments, vision data analysis component 212 works with machine learning system 134 to improve the accuracy and depth of its analysis. For example, the component may provide segmented images to machine learning system 134 for further analysis, such as detecting signs of diseases like glaucoma or macular degeneration. In this case, vision data analysis component 212 first preprocesses the video data to ensure that it is in a format compatible with the machine learning models, applying noise reduction and image stabilization techniques as necessary. The results from machine learning system 134 may be used to guide treatment decisions, such as adjusting the focal point or intensity of laser treatments during procedures.

In an embodiment, vision data analysis component 212 is operable to generate annotations and overlays that highlight specific anatomical features or potential abnormalities in the eye. These annotations can be transmitted to data presentation engine 210 for display on mixed reality device(s) 140. For instance, if vision data analysis component 212 detects abnormal retinal layers or irregularities in corneal thickness, these areas can be highlighted with visual cues, such as color-coded regions, to assist medical professionals during examinations or treatments. In certain cases, the system may automatically prompt the doctor to focus on specific areas of concern, guiding them during both diagnostics and interventions.

In various embodiments, vision data analysis component 212 applies temporal analysis techniques to track changes in eye anatomy over time. This is particularly useful in monitoring progressive conditions like diabetic retinopathy or glaucoma. By comparing current 3D video data with historical data stored in 3D eye data datastore 133, vision data analysis component 212 can detect subtle changes in eye structure and provide longitudinal assessments. For instance, it may detect a thinning of the retina over time, which could indicate disease progression. This historical analysis may also be used to guide ongoing treatment decisions, helping the system identify when a procedure should be adjusted based on changes in the patient's eye anatomy.

In another embodiment, vision data analysis component 212 includes feature extraction capabilities that allow it to identify specific landmarks within the eye, such as the fovea, macula, or optic disc. These features are critical for various diagnostic procedures, and vision data analysis component 212 can accurately pinpoint their location within the 3D video data. For example, in cases where the system is used to assess macular degeneration, vision data analysis component 212 is operable to focus on the macula, extracting detailed measurements and identifying any anomalies. In certain treatment scenarios, such as during a LASIK procedure, the system may use these extracted features to guide the laser alignment, ensuring precise targeting of the corneal tissue.

Vision data analysis component 212 is operable to generate diagnostic data that can be used by other components within integration system 136, such as machine learning system 134 and diagnosis component 216. For example, the diagnostic data generated by vision data analysis component 212 could be used as input to machine learning models designed to predict disease progression or to assist in creating a patient-specific treatment plan. In certain embodiments, the system uses this data to inform real-time adjustments during treatment, ensuring that procedures are responsive to ongoing changes in eye anatomy detected by the vision data analysis component.

In certain embodiments, vision data analysis component 212 can also perform real-time analysis of the 3D video data during the examination or treatment process. As new data is received from vision intake system 132, vision data analysis component 212 processes the video frames in real-time, ensuring that up-to-date analysis is available for immediate decision-making by the medical professional. This real-time capability is essential for providing live feedback during procedures, such as cataract surgeries, laser treatments, or robotic-assisted interventions, where the physician may need to make on-the-spot adjustments based on the visualized data.

Vision data analysis component 212 is operable to handle different image formats, resolutions, and frame rates, depending on the capabilities of the vision intake system 132 and the requirements of the specific examination or treatment. For example, it may process high-resolution images for detailed diagnostic purposes or lower-resolution data for faster, real-time analysis. In some embodiments, vision data analysis component 212 can dynamically adjust its processing techniques based on available bandwidth and computational resources, ensuring optimal performance across different operating environments. For treatment guidance, the system may adjust its data processing to ensure that real-time analysis is prioritized for critical procedures.

There are alternative implementations of vision data analysis component 212 that could be employed depending on system requirements or specific use cases. One alternative could involve the use of cloud-based analysis, where the raw 3D video data is transmitted to a remote server for processing, reducing the computational load on local devices. This cloud-based approach would enable the system to scale more easily and handle larger datasets. Another alternative could involve distributed processing, where different components of vision data analysis component 212 operate on separate devices or servers, allowing for parallel processing of 3D video data to improve efficiency and reduce processing times. In either scenario, the system could also integrate external diagnostic or treatment tools to enhance the capabilities of the vision data analysis component.

In another embodiment, vision data analysis component 212 can be integrated with external image analysis tools or third-party diagnostic software, allowing for more specialized or complex analyses. For example, it could interface with a dedicated retinal analysis tool for assessing specific conditions like retinopathy of prematurity or interface with corneal topography systems to assist in LASIK surgeries. Vision data analysis component 212 would ensure that the external data is properly synchronized and integrated with the existing 3D video data for a comprehensive view of the patient's eye health.

In various embodiments, vision data analysis component 212 includes built-in redundancy and error-checking mechanisms to ensure the accuracy and reliability of the processed data. For example, it may use checksum verification or cross-reference its results with historical data to validate the findings. This ensures that the diagnostic outputs generated by vision data analysis component 212 are both accurate and reliable, minimizing the potential for misdiagnosis. Additionally, the system may apply these mechanisms during treatment to ensure that live feedback and adjustments are consistently accurate and responsive.

Vision acquisition enhancement component 213 is operable to optimize and enhance the quality of the 3D video data captured by vision intake system 132. More specifically, vision acquisition enhancement component 213 performs real-time image adjustments and enhancements to improve the clarity, stability, and precision of the captured video data. In certain embodiments, vision acquisition enhancement component 213 integrates with robotic systems, such as servo-controlled slit lamps, motorized computer vision-driven autofocus, allowing the component to dynamically adjust the position, focus, and angle of the camera or other treatment devices based on real-time feedback from the 3D video data and computer vision.

For example, in various embodiments, vision acquisition enhancement component 213 applies a series of image processing techniques to the incoming 3D video data. These techniques may include focus adjustment, brightness and contrast balancing, and noise reduction. For example, if the 3D video feed captures an image with poor contrast due to insufficient lighting during the eye examination, vision acquisition enhancement component 213 can dynamically adjust the contrast levels to ensure that anatomical details are clearly visible. Similarly, the component can adjust the sharpness and focus of the video to ensure the regions of interest, such as the retina or cornea, are clearly defined. In certain embodiments, vision acquisition enhancement component 213 is operable to control servo mechanisms and zoom lens focus that physically adjust the camera's focus and position, ensuring that the region of interest remains centered and in focus during both diagnostic and treatment procedures.

In an embodiment, vision acquisition enhancement component 213 is operable to detect and compensate for motion artifacts. For instance, if a patient involuntarily moves during the examination, the component can stabilize the captured 3D video data by applying motion correction algorithms. These algorithms analyze the movement and apply corrections to reduce the blurring or misalignment that might otherwise compromise the accuracy of subsequent analysis. Additionally, the component can automatically adjust the position of the robotic slit lamp or other servo-controlled devices to re-align with the patient's eye after any movement, maintaining optimal focus and alignment for both diagnostic imaging and treatment.

Additionally, vision acquisition enhancement component 213 is operable to improve the resolution of the captured 3D video data using super-resolution techniques. In certain embodiments, the component may apply an interpolation algorithm to increase the resolution of lower-quality video feeds, enabling higher-quality visualizations without the need for additional hardware. This is particularly useful in scenarios where the eye structures being examined are small or intricate, such as the detection of microaneurysms in the retina.

In some embodiments, vision acquisition enhancement component 213 may apply specialized filters to emphasize specific anatomical features of the eye. For example, a filter could be applied to highlight the edges of the optic nerve or the boundaries between different retinal layers, making it easier for subsequent systems to detect and analyze those structures. This preprocessing step helps vision data analysis component 212 and machine learning system 134 perform more accurate diagnostics by improving the overall quality of the input data. Additionally, vision acquisition enhancement component 213 can adjust the camera's focus and field of view in response to feedback from machine learning system 134, ensuring that the treatment device, such as a laser, remains precisely aligned with the target area throughout the procedure.

Vision acquisition enhancement component 213 is operable to integrate real-time feedback from other components, such as machine learning system 134. For example, machine learning system 134 may detect suboptimal video capture conditions, such as excessive noise or poor lighting, and transmit corrective instructions to vision acquisition enhancement component 213 to improve the video quality. This feedback loop ensures that the highest quality data is captured and processed throughout the examination. In some embodiments, machine learning system 134 can also guide adjustments to the camera or treatment device's positioning and focus, ensuring that the system remains centered on critical structures during both diagnosis and treatment.

In certain embodiments, vision acquisition enhancement component 213 supports manual adjustments made by medical professionals. For instance, the component may allow a doctor to manually adjust the focus, brightness, or contrast of the 3D video feed using a control panel, voice commands, or even hand gestures within a mixed reality interface. This feature provides flexibility and control during the examination process, allowing the medical professional to fine-tune the video feed or treatment device positioning in real-time based on their expertise and preferences.

In various embodiments, vision acquisition enhancement component 213 is operable to enhance the 3D video data for both live viewing and subsequent analysis. The enhanced data can be stored in vision processing data store 224 or transmitted directly to vision data analysis component 212 for immediate processing. This dual functionality ensures that the data is ready for immediate diagnostic evaluation and for future reference in follow-up examinations or comparative analysis. Moreover, the component's ability to control the position and focus of robotic treatment devices based on real-time data enhances the precision of procedures, such as laser surgeries, by continuously maintaining alignment with the target area of the eye.

Data mapping component 214 is operable to map 3D video data captured by vision intake system 132 to relevant contextual information stored in patient clinical data store 220. More specifically, data mapping component 214 aligns the regions of the eye identified in the 3D video data with corresponding medical records, diagnostic results, and other contextual data, enabling a comprehensive view of the patient's eye health during the examination.

In various embodiments, data mapping component 214 utilizes metadata, timestamps, and spatial coordinates derived from the 3D video data to accurately match the video segments with the relevant medical data. For example, if the 3D video data captures the optic nerve region, data mapping component 214 can retrieve historical diagnostic information related to optic nerve conditions, such as glaucoma or optic neuritis, and map this information to the live video feed. This contextual information is then displayed in the mixed reality environment through data presentation engine 210.

In an embodiment, data mapping component 214 applies a combination of image recognition algorithms and spatial mapping techniques to perform the mapping process. For instance, the component may use computer vision techniques to identify specific anatomical structures within the 3D video data, such as the retina or cornea, and then cross-reference these regions with previously stored clinical data in patient clinical data store 220. This cross-referencing ensures that the correct contextual data is associated with the corresponding anatomical region being examined.

Data mapping component 214 is also operable to handle multiple sources of contextual information. For example, the component can map not only historical patient data but also real-time diagnostic results generated by machine learning system 134. In this scenario, data mapping component 214 would associate live diagnostic outputs, such as measurements of corneal thickness or retinal detachment, with the corresponding regions in the 3D video feed. This ensures that the most up-to-date diagnostic data is readily accessible and mapped to the appropriate eye structures during the examination.

In certain embodiments, data mapping component 214 is operable to map 3D video data to external data sources, such as third-party medical databases or research repositories. For example, the system may pull data from external databases that provide additional insights on rare eye conditions or clinical trial results. Data mapping component 214 integrates this external information with the patient-specific data, presenting it alongside the 3D video data in the mixed reality environment for a more comprehensive diagnostic view.

In an embodiment, data mapping component 214 is operable to incorporate predictive models into the mapping process. For instance, machine learning models trained on large datasets of patient eye scans may predict the likelihood of future conditions such as macular degeneration based on current examination data. Data mapping component 214 can map these predictive insights to the specific regions of the eye being examined, providing the medical professional with not only current diagnostic data but also future risk assessments.

In various embodiments, data mapping component 214 supports dynamic data updates during the examination process. As new data is captured by vision intake system 132 or new diagnostic results are generated by machine learning system 134, data mapping component 214 updates the mapped data in real-time. This ensures that the most current information is always available to the medical professional during the examination.

In certain embodiments, data mapping component 214 applies data validation techniques to ensure the accuracy and consistency of the mapped information. This may involve cross-referencing the mapped data against multiple data sources or performing integrity checks to verify that the correct contextual data is associated with the 3D video feed. For example, if the system detects discrepancies between historical data and real-time diagnostic results, data mapping component 214 can flag these inconsistencies for further review by the medical professional.

In various embodiments, data mapping component 214 allows for manual input from the medical professional. For instance, the doctor may manually select a region of the eye within the 3D video feed and specify the type of contextual information they would like to see mapped to that region. This manual mapping feature provides flexibility in cases where automated mapping may not capture all relevant data or when the doctor wishes to explore specific areas of interest.

Data mapping component 214 is operable to store and organize the mapped data for future reference. In certain embodiments, the mapped data is stored in a structured format that allows for efficient retrieval and comparison in subsequent examinations. For example, the system may store the mapped data alongside timestamps and patient identifiers, enabling longitudinal analysis of the patient's eye health over time.

Diagnosis component 216 is operable to generate diagnostic outputs based on the 3D video data captured by vision intake system 132 and contextual information retrieved from patient clinical data store 220. More specifically, diagnosis component 216 applies machine learning models and diagnostic algorithms to assess the state of the patient's eye and identify potential medical conditions or abnormalities. These diagnostic outputs may include measurements, classifications, and predictions related to various eye health metrics.

In various embodiments, diagnosis component 216 works in conjunction with machine learning system 134 to process the 3D video data and other relevant data sources. For example, diagnosis component 216 can analyze the segmented regions of the eye (such as the retina, cornea, or optic nerve) and apply diagnostic algorithms to detect structural anomalies, such as retinal detachment, macular degeneration, or corneal dystrophies. The outputs generated by diagnosis component 216 can include both quantitative measurements, such as retinal thickness or intraocular pressure, and qualitative assessments, such as the classification of observed abnormalities.

In an embodiment, diagnosis component 216 leverages trained machine learning models, such as convolutional neural networks (CNNs) or other deep learning architectures, to automatically classify specific conditions based on the visual data. For instance, diagnosis component 216 can use these models to identify signs of diabetic retinopathy by recognizing patterns in the retinal vasculature. The classification results may then be used to assist the doctor in making a diagnosis or determining the appropriate treatment.

Diagnosis component 216 is operable to generate both real-time diagnostic outputs and post-examination reports. In real-time, the component analyzes the incoming data from vision intake system 132 and provides immediate feedback to the medical professional during the examination. For instance, as the 3D video of the patient's eye is being displayed on mixed reality device(s) 140, diagnosis component 216 can overlay the diagnostic outputs, such as measurements of corneal thickness or optic nerve health, directly on the mixed reality display.

In certain embodiments, diagnosis component 216 supports interactive diagnostics. For example, the medical professional can manually adjust the diagnostic parameters or select specific regions of the eye for more focused analysis. Diagnosis component 216 would then reprocess the data based on the new parameters and provide updated diagnostic outputs. This interactive capability allows for more tailored diagnostics and provides the flexibility to respond to specific areas of concern during the examination.

Diagnosis component 216 is also operable to integrate multiple diagnostic models into its analysis. For example, it may apply different machine learning models for different eye structures or conditions, such as using one model to detect glaucoma and another model to assess cataract formation. The diagnostic outputs from these models are then combined and presented in a unified format to the medical professional. In certain embodiments, diagnosis component 216 also supports the integration of external diagnostic tools or models, allowing for enhanced diagnostic capabilities beyond the native models within the system.

In an embodiment, diagnosis component 216 can generate predictive analytics based on historical patient data and machine learning predictions. For example, the component can analyze trends in a patient's eye health over time, such as progressive thinning of the retinal layers, and use predictive models to estimate the likelihood of future complications, such as the development of macular degeneration. These predictive insights can assist in early intervention and treatment planning.

In certain embodiments, diagnosis component 216 applies multiple diagnostic techniques, including both rule-based algorithms and machine learning models, to enhance the accuracy of the diagnostic outputs. For example, a rule-based algorithm may assess intraocular pressure readings against established clinical thresholds, while a machine learning model simultaneously analyzes the same data to detect less obvious patterns indicative of potential glaucoma.

Diagnosis component 216 is operable to store the diagnostic outputs in diagnostic data datastore 135 for future analysis and retrieval. These outputs may be organized based on patient identifiers, specific conditions, or examination timestamps, enabling medical professionals to track the progression of eye health over time. For instance, stored diagnostic results may be used to compare a patient's retinal health over multiple visits, helping to identify any worsening conditions or responses to treatment.

In various embodiments, diagnosis component 216 is responsible for validating the diagnostic outputs by cross-referencing them with known medical standards or clinical guidelines. For example, if the component identifies a condition such as glaucoma, it can validate the diagnostic result by comparing the intraocular pressure measurements and optic nerve assessments against established clinical thresholds for glaucoma diagnosis. This ensures that the diagnostic outputs are not only accurate but also clinically relevant.

In certain embodiments, diagnosis component 216 supports real-time updates and notifications. For example, if a new abnormality is detected during an ongoing examination, diagnosis component 216 can immediately notify the medical professional, ensuring that no important diagnostic insights are missed. These notifications can be displayed in the mixed reality environment or delivered to the medical professional via other user devices 130.

Diagnosis component 216 is operable to interact with data mapping component 214 to ensure that the diagnostic outputs are correctly mapped to the relevant regions of the eye. For instance, if the component detects macular degeneration in the patient's retina, it ensures that this diagnostic result is mapped to the corresponding region of the 3D video data for display in the mixed reality environment.

In certain embodiments, diagnosis component 216 applies encryption techniques to secure the diagnostic outputs, particularly when transmitting sensitive patient data across networks or to external devices. This ensures that the diagnostic information remains confidential and protected from unauthorized access.

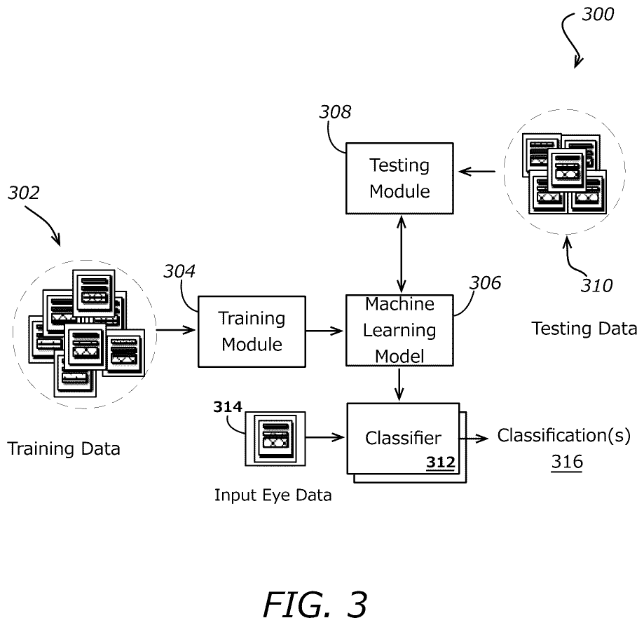

FIG. 3 illustrates an example classification pipeline 300 for processing and classifying eye examination data in accordance with various embodiments. In this example, a set of input eye data 302 is collected from the vision intake system 132 or other appropriate system or datastore and processed to train one or more machine learning models or neural networks 306. These models are operable to classify different anatomical structures of the eye, such as retinal layers, corneal thickness, or optic nerve contours, as well as to identify potential medical conditions.

In various embodiments, input eye data 302 may include 3D video segments, optical coherence tomography (OCT) scans, fundus photographs, or other diagnostic imaging data. This data is used as training data to develop machine learning models, including convolutional neural networks (CNNs) and deep neural networks, to improve the accuracy and speed of diagnosing eye conditions. The eye data can be collected from multiple sources, such as clinical examination records or real-time capture during a patient's visit.

The training data 302 is annotated with ground truth labels indicating specific eye regions or medical conditions, such as macular degeneration or glaucoma. This labeled data serves as a foundation for training machine learning models to recognize similar patterns in new, unseen data. For instance, training data could include labeled segments representing different regions of the retina, allowing the machine learning model 306 to learn the visual characteristics associated with healthy or abnormal retinal structures.

Once the training data has been processed, the data is fed into a training module 304. The training module 304 is responsible for training the machine learning model 306 using supervised learning techniques, wherein the network learns to associate specific input features with corresponding classifications. For example, the training module 304 may train the machine learning model 306 to detect and classify abnormalities such as retinal detachment, optic nerve atrophy, or diabetic retinopathy based on the provided eye data.

In certain embodiments, training data 302 is divided into training and testing subsets. The testing module 308 uses a separate set of testing data 310 to evaluate the performance of the trained model. The testing data 310 includes previously unseen eye images and diagnostic results, which the machine learning model 306 has not been trained on. By comparing the model's predictions to the actual diagnostic results, the system can assess the model's accuracy and refine its parameters.

If the testing results meet the required accuracy thresholds, the machine learning model 306 is deployed into the classification pipeline as classifier 312. The classifier 312 is operable to analyze new input eye data 314 and generate classifications 316 in real-time or during post-capture analysis. These classifications may include anatomical segmentations (e.g., identifying specific layers of the retina) or diagnostic labels (e.g., detecting signs of glaucoma).

In various embodiments, classifier 312 may generate multiple classifications based on different regions of the eye or multiple diagnostic algorithms. For instance, the system may classify one region of the retina as showing early signs of macular degeneration, while another region may be flagged for signs of optic nerve damage. The classifier 312 continuously updates its models as new data is processed, improving its diagnostic capabilities over time.

The classification pipeline 300 is operable to integrate input data from various imaging devices, such as OCT scanners, fundus cameras, and 3D imaging systems. The system can process large volumes of eye data and use deep learning techniques, such as convolutional neural networks (CNNs) and recurrent neural networks (RNNs), to provide accurate and real-time classifications.

In alternative embodiments, the classification pipeline 300 could be modified to support unsupervised learning techniques, such as generative adversarial networks (GANs), which do not rely on pre-labeled training data. Instead, GANs generate synthetic eye data to train the model, reducing the reliance on manually annotated datasets.

The classification pipeline can be used across different examination scenarios, allowing for dynamic analysis of eye health based on a variety of input data types. Additionally, this system supports continuous learning, where new input data can be used to further refine and improve the accuracy of the diagnostic models.

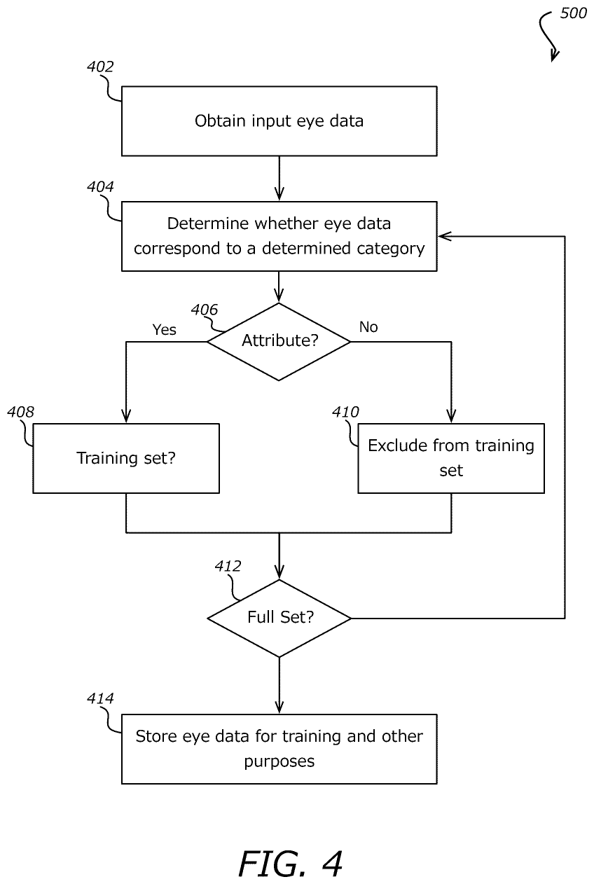

FIG. 4 illustrates an example process 400 for determining training data that can be utilized in accordance with various embodiments.

In this example, a set of eye data is obtained at step 402 for analysis. This data can be obtained from the vision intake system 132, historical medical records, or third-party sources. In certain embodiments, the eye data can be from a catalog maintained by a healthcare provider or a medical database, among other such options. The data can include 3D video data, clinical measurements, or diagnostic results associated with specific regions of the eye, such as the cornea, retina, or optic nerve.

For at least some of the eye data, such as a randomly selected subset or other determined data samples, information associated with the data can be used to determine at step 404 whether the eye data corresponds to a determined category or includes particular attributes for which a machine learning model 306 is to be trained. This can include, for example, a specific category such as corneal thickness, retinal abnormalities, or intraocular pressure. The data attributes can include diagnostic measurements, image patterns, or classifications applied to the eye data during the preprocessing stages.

If it is determined at step 406 that the eye data exhibits the attribute for a particular category, then that data can be added at step 408 to the training set. For example, if the system identifies the data as representing an abnormality in the optic nerve, it can be tagged and incorporated into the set of data used to train machine learning models related to optic nerve diseases. If the data does not exhibit the required attributes, it can be excluded at step 410 from the training set.

At step 412, a check is made to determine whether a full training set has been obtained. A full set may be defined by various criteria, such as reaching a predefined number of data samples or achieving diversity in the types of eye regions represented. If the set is complete, the eye data can be stored at step 414 for training purposes, where it can be used to improve the accuracy of machine learning models within the system, such as machine learning model 306. Otherwise, the process can continue until a full set is obtained, or until all of the relevant eye data is analyzed or another stop condition is satisfied.

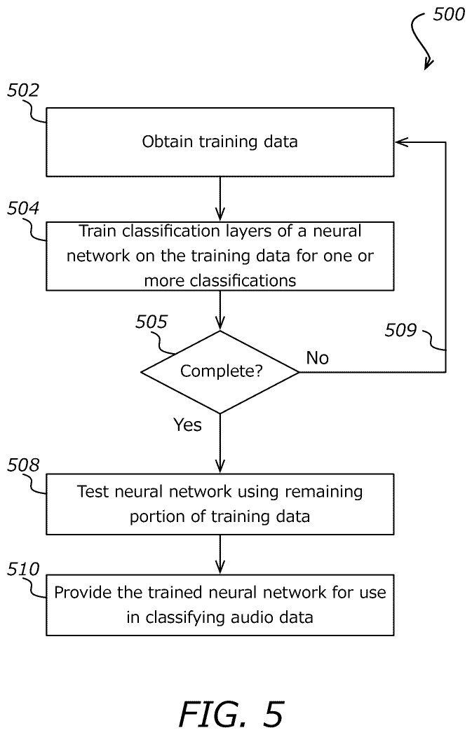

FIG. 5 illustrates an example process 500 for training a machine learning model 306 in accordance with various embodiments. The process begins by obtaining a set of training data 502, which can be provided as input to the training module of the machine learning system 134. In this case, the training data includes 3D video data and relevant diagnostic or contextual information, as described earlier. For example, this could involve historical patient data such as past 3D imaging and associated diagnostic results or medical scans related to the regions of the eye under analysis.

The next step in the process involves training 504 the machine learning model 306 using the training data obtained. The machine learning model can be trained to recognize patterns or features within the 3D video data that correspond to specific diagnostic classifications or abnormalities in the eye. For instance, machine learning model 306 may use convolutional neural networks (CNNs) to identify retinal layers or detect signs of macular degeneration based on the training data provided. This training allows the model to accurately classify specific anatomical features and generate diagnostic insights when analyzing new 3D video data captured by vision intake system 132.

Once the initial training is complete, the process moves to a testing phase 508, where a portion of the training data is reserved for testing. This ensures that the machine learning model's accuracy can be evaluated before it is deployed. In this phase, the machine learning system 134 analyzes the remaining portion of the training data, generating diagnostic outputs for comparison with ground-truth labels or expected results. The results of this testing phase are used to determine whether the model's accuracy meets the required thresholds.

If the machine learning model 306 passes the testing phase with acceptable accuracy, the process completes 510, and the trained model is deployed for use during real-time examinations. For example, the trained model may be used to classify new 3D video segments captured during a patient's eye examination and provide diagnostic insights in real-time. These insights can include identifying anomalies, such as corneal dystrophies or optic nerve damage, based on the features learned during training.

However, if the results of the testing phase indicate that the model's performance does not meet the necessary standards, the process can return to step 504, where additional training iterations are performed. This cycle of training and testing continues until the machine learning model 306 achieves the desired level of diagnostic accuracy.

In various embodiments, the machine learning model 306 can incorporate various learning techniques, such as supervised learning using labeled training data or unsupervised learning to discover hidden patterns within the 3D video data. Additionally, machine learning system 134 may utilize generative models to augment the training dataset, improving the model's ability to handle edge cases or rare diagnostic conditions.

Once the model is trained and verified, it can be used in conjunction with other system components, such as vision data analysis component 212 and data presentation engine 210, to enhance the diagnostic capabilities of the system. For example, the trained model can help improve the precision of real-time diagnostic outputs by providing accurate, automated classifications based on the 3D video data obtained from vision intake system 132.

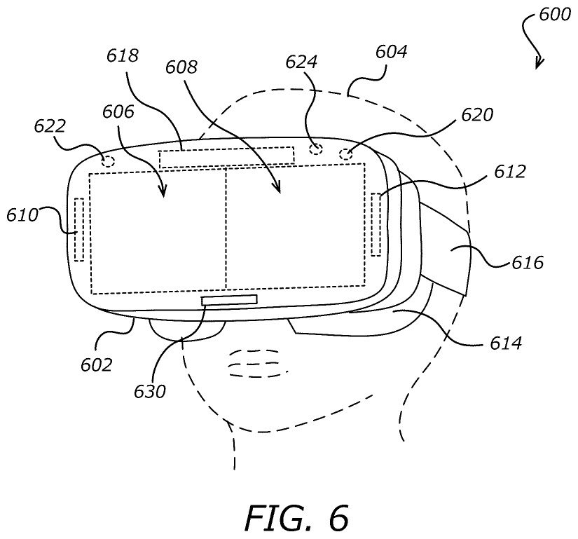

FIG. 6 illustrates an example mixed reality device 600 that can be utilized in accordance with various embodiments of the invention. Various types of devices, such as smart glasses, goggles, and other virtual and/or augmented reality displays, can be used within the scope of the invention. In this example, mixed reality device 600 is operable to receive and display 3D video data, contextual information, and diagnostic results from integration system 136, presenting these data streams in a virtual or augmented reality environment.

Mixed reality device 600 includes various components designed for user interaction, data visualization, and environmental awareness. For instance, mixed reality device 600 includes left eye display 602 and right eye display 604, which present stereoscopic 3D visuals, such as the 3D eye video captured by vision intake system 132. These displays may include AMOLED or LED technology and are capable of rendering high-definition, volumetric visualizations of anatomical structures, such as the retina or optic nerve. The mixed reality display can include overlays of diagnostic data, such as retinal thickness or intraocular pressure, facilitating real-time examination.

Mixed reality device 600 can include one or more motion and orientation sensors 606, which can provide data on the device's position and movement. In certain embodiments, these sensors can include accelerometers, gyroscopes, magnetometers, and other components to detect changes in the user's head orientation, allowing the displayed 3D video to adjust to the user's point of view. This enables an immersive and responsive examination experience, where the doctor can move their head to view different angles of the patient's eye anatomy.

In an embodiment, mixed reality device 600 includes one or more external cameras 608 operable to capture environmental data, including real-world objects or medical instruments around the user. These external cameras can assist with spatial awareness, ensuring the augmented reality interface adapts to the user's physical surroundings. For example, if the user is handling a medical tool, the system may adjust the mixed reality interface to incorporate the tool into the visualization.

Additionally, mixed reality device 600 includes one or more internal cameras 610 for capturing the user's gaze direction, facial expressions, or other biometric data. These internal cameras may be operable to track the position of the user's pupils, providing eye-tracking capabilities that can enhance the user experience by enabling the user to control certain interface elements using their gaze. For instance, the user may focus their gaze on a specific region of the eye displayed in the 3D video, prompting the system to zoom in on that region or provide additional contextual information.

Mixed reality device 600 is operable to accept various forms of user input, such as voice commands, hand gestures, and physical controllers. For example, the device may include one or more microphones 612 for capturing voice input from the user. A doctor may use voice commands to control the mixed reality interface, such as requesting specific diagnostic results, switching between different examination views, or adjusting the displayed data.

In another embodiment, mixed reality device 600 is operable to detect hand gestures using integrated sensors or external cameras. These hand gestures can be used to interact with the data displayed in the mixed reality environment. For instance, the doctor could use a pinching gesture to zoom in on a specific area of the 3D video or a swiping gesture to switch between different data layers, such as toggling between 3D video and contextual information overlays.

In various embodiments, mixed reality device 600 includes communication components 614 that are operable to transmit and receive data from integration system 136. These communication components can include wireless technologies such as Wi-Fi, Bluetooth, or other appropriate protocols for transmitting the 3D video data, diagnostic results, and contextual information from the system to the mixed reality device 600.

To ensure smooth operation and high-quality display, mixed reality device 600 can include display circuitry 616, which manages data rendering, display updates, and other processing tasks related to the presentation of 3D video and diagnostic information. This circuitry can include processors, graphics processors, and memory components that are operable to process the incoming data and render it in a way that is optimized for the mixed reality device.

Additionally, in certain embodiments, mixed reality device 600 is equipped with input and output ports 618, such as USB or HDMI, to allow for wired data transmission between the device and external systems. These ports can also be used to connect peripheral devices, such as external controllers or additional sensors.

Mixed reality device 600 is further operable to support interactive features, enabling medical professionals to manipulate the 3D video and diagnostic data in real time. For instance, using voice commands, hand gestures, or external controls, the user can rotate the 3D eye model, zoom in on specific structures, or toggle between different diagnostic overlays. These features improve the examination process by providing the user with dynamic control over the mixed reality interface.

In an embodiment, mixed reality device 600 continuously receives updates from integration system 136, ensuring that the displayed 3D video and diagnostic information are synchronized and up to date. This ensures that the medical professional has access to the most recent data during the examination, allowing for real-time analysis and decision-making.

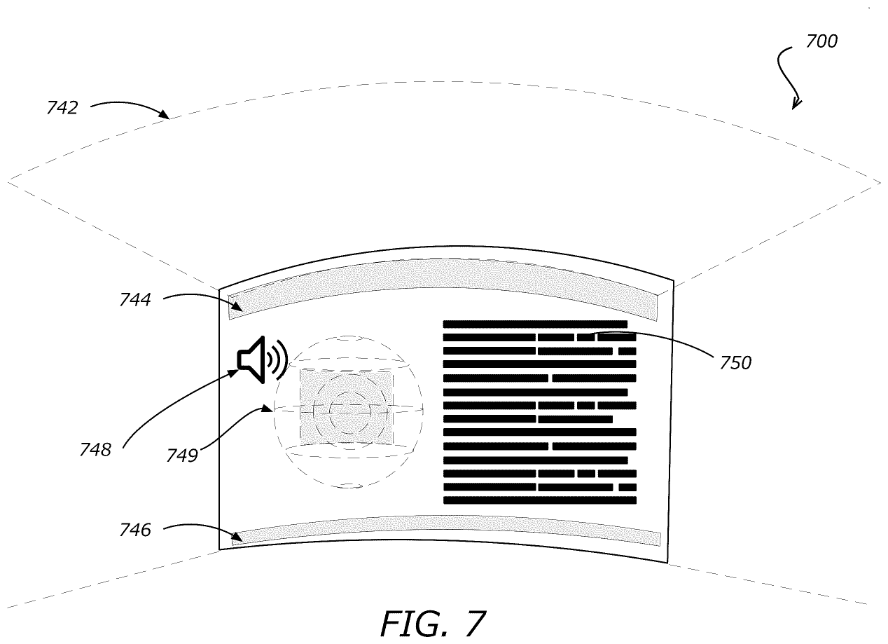

FIG. 7 illustrates an example mixed reality interface 700 presented to a user, such as a doctor, during an eye examination using mixed reality device(s) 140. The mixed reality interface allows the user to view and interact with multiple layers of data, including 3D visualizations of the eye, diagnostic information, and patient-specific data, within a virtual or augmented reality environment.

The interface 700 includes several elements for the examination and treatment process. For example, visible area 742 represents the primary viewport through which the user observes the content displayed by the mixed reality device(s). This viewport may include layered content, with the ability to reposition, resize, or update the various visual elements depending on user preferences and system configurations. The visible area may display overlapping media layers, allowing the user to toggle between different types of information, such as the 3D eye visualization and diagnostic data.

Header section 744 of the interface 700 provides a location for displaying information relevant to the examination. This section can include time information, orientation or positional data, user profile information, notifications, or other relevant data that does not interfere with the central examination process. The header section remains accessible throughout the examination without distracting from the primary display area, enabling the user to stay informed about system status or environmental conditions while focusing on the patient's eye.

Footer section 746 similarly offers a persistent, non-intrusive space for additional controls and data. The footer can contain interface elements such as access to different categories of content, options for initiating new commands, and controls for interacting with other system components. For instance, the user may use this section to control the visibility of different eye layers, initiate zoom features, or manage treatment guidance provided by the system.

Graphical icon 748 in the interface represents a control option, such as adjusting the volume of system notifications or other auditory inputs. The user can modify the system's audio settings using gestures, voice commands, or a controller. In some embodiments, graphical icon 748 may serve as a multi-functional control, providing access to other system preferences, including display brightness or notification settings. This customization allows the user to tailor the interface to their needs without leaving the examination workflow, ensuring a smooth experience while performing a detailed eye analysis.

A representation of the eye 749 is shown, which can display both internal and external sections of the eye, such as the cornea, retina, or optic nerve. More than one visualization of the eye may be presented simultaneously. The system allows the user to zoom in and out of different sections of the eye and to outline specific regions using hand gestures. For instance, the user may use a gesture to trace the boundaries of the retina, prompting the system to automatically zoom in and provide additional diagnostic overlays for that region. The system may also use colors or other overlays to highlight sections of the eye requiring further investigation, such as areas flagged by machine learning system 134 for abnormalities or regions of interest where the CV-guided treatment algorithms have detected a need for focus adjustments during procedures. Additionally, the interface can dynamically suggest regions to investigate based on real-time analysis, guiding the user's focus to key areas.

Diagnostic data, patient history, and other relevant information are presented in a data section 750. This section displays real-time diagnostic outputs from machine learning system 134, such as assessments of intraocular pressure, corneal thickness, detected abnormalities, and potential treatment recommendations. The system can overlay this data directly onto the 3D visualization of the eye, creating a comprehensive view that integrates both visual and diagnostic information. Additional overlays may be employed to emphasize critical sections of the eye, guiding the user's attention to areas that require immediate review or treatment. For example, the system may alert the user to potential focus drift during treatment and suggest corrective actions to maintain precision.

The user has control over the display of different “layers” of the eye. For example, they can choose to hide or display certain anatomical layers, such as focusing only on the retina while temporarily hiding the cornea or optic nerve. In certain embodiments, the system automatically guides the user through the examination by bringing different aspects of the eye into focus based on pre-programmed diagnostic workflows or system-detected abnormalities. For instance, the system may automatically zoom in on the optic nerve if signs of glaucoma are detected, or prompt the user to examine the cornea if irregularities in thickness are observed. Additionally, during both examination and treatment, the system dynamically adjusts focus and centering, driven by CV algorithms, ensuring that the targeted region remains centered and clear. For example, during laser treatments, the system can adjust the position and focus in real-time based on detected anatomical movements or changes in the eye's surface, improving procedural precision.

In addition to the automatic guidance, the interface 700 allows the user to manually adjust the display according to their needs. The doctor may rearrange the layout of the displayed data, resize the 3D eye visualization, or select which diagnostic metrics or treatment guidance elements are prioritized in the interface. The system's flexibility ensures that all relevant data is easily accessible, improving the workflow and enhancing both diagnostic and treatment capabilities during the examination.

The interface can display additional graphical icons or controls based on the user's input. For example, the user may enable annotations, measurement tools, or other overlays directly on the 3D visualization. The interface also supports interactive features, where the user can engage with specific data points using hand gestures, voice commands, or a controller. For instance, the doctor can mark specific regions of the eye for further analysis or initiate additional tests or treatments based on real-time data. Treatment guidance provided by the system may include visual cues or annotations that help the user maintain optimal focus and positioning during procedures such as laser treatments, using CV-based feedback to dynamically adjust the display.

The system continuously updates the displayed information in real-time as new diagnostic data or 3D video segments are processed. This ensures that the user always has the most up-to-date information throughout the examination, enhancing both decision-making and patient care. Additionally, the system can be configured to remember user preferences, prioritizing commonly used diagnostic metrics or treatment views based on the doctor's previous examinations and treatment procedures.

In various embodiments, the mixed reality interface 700 may incorporate further customizations, such as the integration of external data sources or third-party diagnostic tools. This allows for additional medical information, such as pharmaceutical data, genetic information, or external treatment guidelines, to be displayed alongside the 3D visualization of the eye. The system ensures that this data is formatted appropriately and synchronized with the existing patient information and diagnostics, aiding in both diagnosis and treatment planning.

In some embodiments, the interface also supports interaction modes, such as a presenter mode, where multiple users can view the same eye examination and treatment data in a collaborative session. This feature is particularly useful in educational or training environments, allowing one user to guide others through the examination and treatment process by controlling the mixed reality environment for all participants.

The system may also provide secure, encrypted transmission of patient data to ensure the confidentiality of sensitive medical information displayed within the mixed reality environment. Additionally, data validation techniques, such as checksums or secure hashing algorithms, can be applied to ensure the accuracy of the transmitted diagnostic and contextual information. During treatment, this ensures that real-time feedback and corrections are accurately reflected in the interface, supporting precise and safe medical procedures.

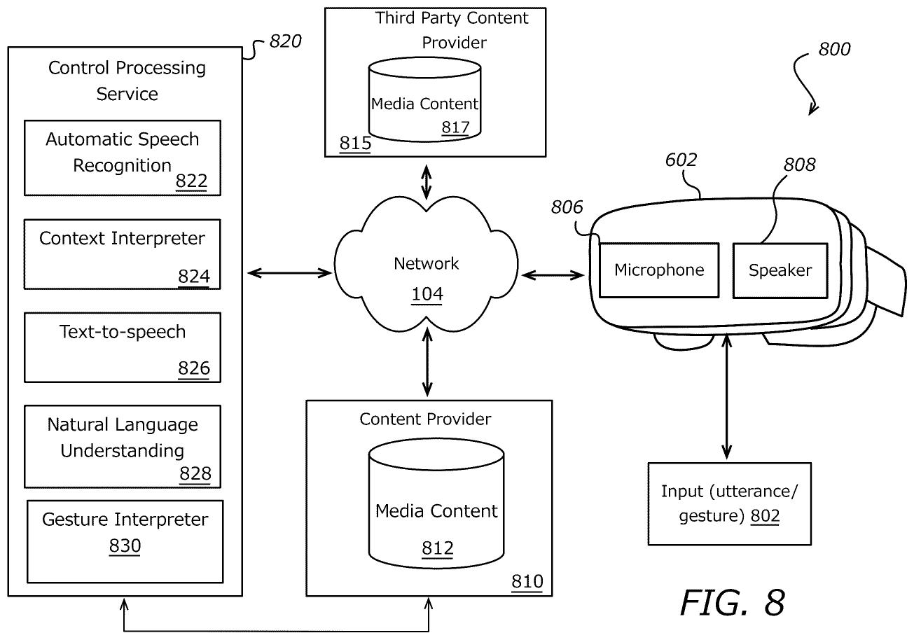

FIG. 8 illustrates an example system 800 for receiving and processing various types of user inputs, including voice commands and hand gestures, in accordance with various embodiments. This system facilitates interaction with media content and diagnostic data during an ophthalmic examination using mixed reality device(s) 140. The system enables hands-free control and dynamic adjustments to the mixed reality environment by incorporating control processing service 820, gesture interpreter 830, and other input recognition services.

In this embodiment, system 800 shows the interaction between a mixed reality device 140 and several system components responsible for processing user input and delivering content. Mixed reality device 140 can include input components such as microphone 806 and gesture recognition sensors (not shown in figure) for receiving user commands in the form of voice utterances or hand gestures. These inputs are processed and interpreted by control processing service 820, which is operable to handle voice commands and gestures, adjusting the system's behavior or content accordingly.

Control processing service 820 manages a series of specialized modules, including automatic speech recognition (ASR) module 822, natural language understanding (NLU) module 828, and gesture interpreter 830. For instance, when a voice command or gesture input 802 is detected, ASR module 822 converts the speech into text, while NLU module 828 interprets the intent of the spoken command. If a gesture is detected, gesture interpreter 830 processes the input to interpret specific actions, such as zooming into a section of the eye or switching between anatomical layers in the mixed reality environment.

The content provider 810 delivers media content and diagnostic information to the mixed reality device(s) 140, based on the commands received from the control processing service 820. For example, the user can speak or gesture to request the display of 3D visualizations of the patient's eye or overlay diagnostic information related to the eye examination. The content provider 810 retrieves the necessary content from media content data store 812 or from third-party content providers 815, which may store additional diagnostic tools or patient history data. Once retrieved, the data is displayed on mixed reality device(s) 140.

In one embodiment, gesture interpreter 830 supports complex hand gestures, allowing users to interact with the 3D visualization without needing to use physical controls. For example, a doctor could use a pinch gesture to zoom in on a particular region of the eye or swipe to navigate between different diagnostic overlays. Gesture interpreter 830 translates these hand motions into system commands that are executed in real-time, ensuring a seamless and intuitive user experience.

The system also supports audio feedback and notifications via speaker 808, allowing users to receive auditory alerts or spoken responses based on their commands. Text-to-speech module 826 within control processing service 820 converts textual data into speech, providing real-time updates or reminders during the examination. For example, the system may notify the user if the diagnostic models detect an abnormality, prompting the user to investigate further.

The communication network 104 connects all components, enabling interaction between the mixed reality device(s) 140, control processing service 820, content provider 810, and third-party content providers 815. The network facilitates the transmission of real-time data, ensuring that user inputs are quickly processed and the corresponding content is promptly displayed or adjusted based on the user's commands.

In various embodiments, system 800 allows for personalized configurations based on user preferences. For instance, the control processing service 820 can remember a doctor's preferred commands and interaction styles, prioritizing specific diagnostic data or adjusting the mixed reality environment automatically according to the user's historical interactions.

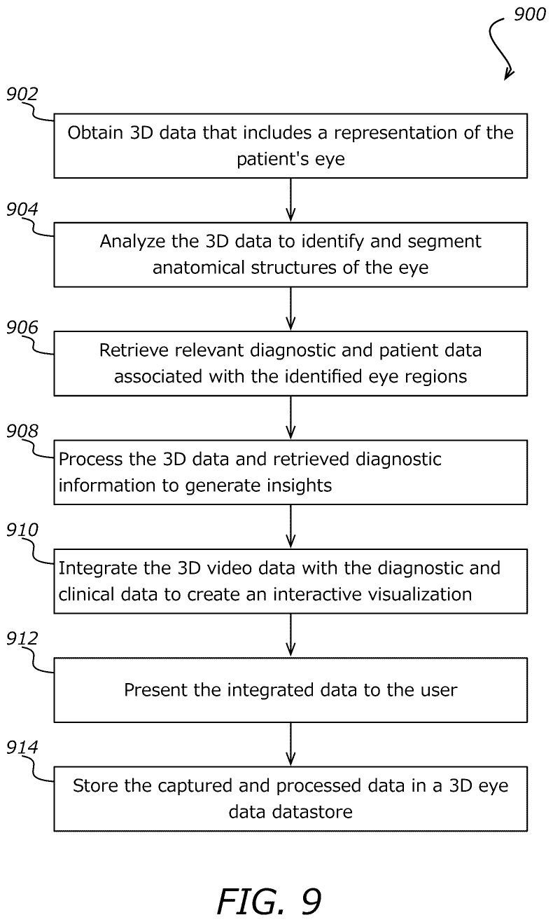

FIG. 9 illustrates an exemplary process 900 for obtaining 3D eye data, analyzing the data, retrieving diagnostic and patient information, and presenting this information in an integrated mixed reality environment in accordance with various embodiments. The process steps may be performed by a system, such as the system described in FIG. 1B, FIG. 2, and FIG. 7, or in association with a different system. The process may comprise additional steps, fewer steps, and/or a different order of steps without departing from the scope of the invention as would be apparent to one of ordinary skill in the art.

At step 902, 3D data (e.g., 3D video data) of the patient's eye is obtained. In various embodiments, the 3D video data may be obtained from vision intake system 132 or another appropriate system. The system captures high-resolution, three-dimensional visual data of the eye, including key anatomical structures such as the cornea, retina, and optic nerve. This data forms the basis for further analysis. In some embodiments, alternative imaging techniques such as 2D video, optical coherence tomography (OCT), or slit-lamp images may be used, depending on the examination's requirements. The captured data is operable to provide detailed visual information, ensuring that critical eye structures are accurately represented.

At step 904, the system analyzes the obtained 3D video data. The system utilizes computer vision algorithms and other appropriate techniques to process the 3D video data, identifying, segmenting, and recognizing different anatomical structures. For example, the system may detect the boundaries of the cornea, optic nerve, and retina, differentiating between layers based on their specific visual properties. The system may also use machine learning models trained on large datasets of eye imagery to automatically classify various eye regions. In certain embodiments, the system is operable to receive manual input from the user to further refine or confirm the region of interest. The user may provide input using gestures, voice commands, or a touchscreen interface, such as manually selecting an area of the retina for further analysis.

At step 906, the system retrieves diagnostic and patient data associated with the identified eye regions. Once the system has segmented and identified the eye region at step 904, it queries diagnostic data datastore 135 and patient clinical data store 220 to obtain relevant medical information, such as historical measurements, previous diagnoses, or test results specific to that eye region. For instance, if the cornea was identified in step 904, the system may retrieve corneal topography measurements or past assessments of corneal thickness. Additionally, the system may retrieve comparative data to analyze trends, such as the progression of retinal thickness over time. In certain embodiments, the system allows the user to input specific commands to retrieve desired data, such as patient records or diagnostic reports associated with previous eye exams.

At step 908, the system processes the 3D video data and the retrieved diagnostic information to generate insights. For example, machine learning system 134 may apply diagnostic models trained on various eye conditions to analyze the identified regions and data. The system may identify abnormalities, such as thinning of the retinal nerve fiber layer or optic nerve damage, based on the combination of 3D visual data and historical diagnostic information. In one embodiment, the system uses algorithms to assess intraocular pressure by analyzing the data changes over time. The diagnostic results generated by the system may include flagged regions that require further attention, numerical metrics like corneal thickness, or recommendations for additional examination or treatment.

At step 910, the system integrates the 3D video data with the retrieved clinical data and diagnostic analysis for display. The data presentation engine 210 assembles the processed 3D video data, diagnostic information, and any retrieved clinical data into a coherent format for display in a mixed reality interface. The system overlays diagnostic information directly onto the 3D visualization, ensuring that the doctor can see both the real-time eye data and relevant medical information in one view. For example, color-coded regions may be used to indicate areas where abnormalities were detected, or numerical metrics may be displayed alongside the corresponding eye structures. In certain embodiments, the system supports dynamic visualization options, such as toggling between different diagnostic overlays or comparing historical data with current examination results.

At step 912, the integrated data is presented to the user, such as a medical professional, via the mixed reality interface provided by mixed reality device(s) 140. The system ensures that the combined 3D video data, diagnostic information, and clinical history are presented in real-time, allowing the user to interact with the data using hand gestures, voice commands, or other input methods. For example, the user may zoom in on specific regions of the eye, such as the retina, or toggle between various diagnostic layers. Additionally, the system may allow the user to rearrange the layout of displayed data or prioritize specific diagnostic metrics. In some embodiments, the system supports voice commands for hands-free navigation during the examination.

At step 914, the system stores the captured and processed data in 3D eye data datastore 133 for future reference. This stored data may include the raw 3D video data, any diagnostic results generated by machine learning system 134, and annotations made during the examination. In an embodiment, the system indexes the stored data according to the specific region of the eye examined, allowing for efficient retrieval in future exams. For instance, the system may enable historical trend analysis, such as tracking changes in corneal thickness or retinal health over time. This functionality provides continuity in patient care and assists in long-term monitoring of eye health.

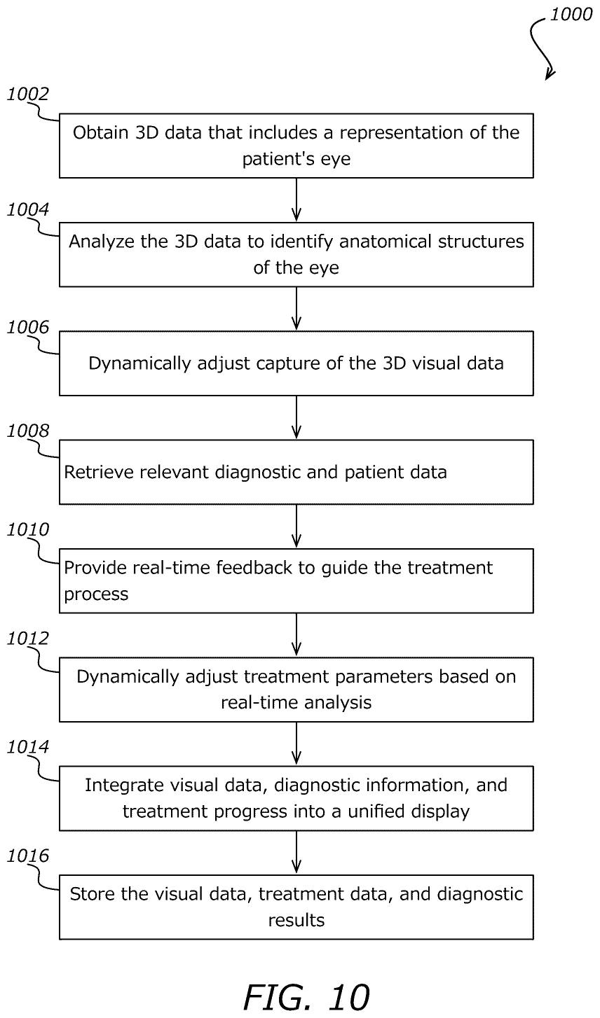

FIG. 10 illustrates an exemplary process 1000 for guiding visual data capture and treatment in an integrated mixed reality environment using computer vision techniques in accordance with various embodiments. The process steps may be performed by a system, such as that described in FIG. 1B, FIG. 2, and FIG. 8, or in association with a different system. The process may comprise additional steps, fewer steps, and/or a different order of steps, without departing from the scope of the invention as would be apparent to one of ordinary skill in the art.

At step 1002, 3D visual data of the patient's eye is obtained, for example, using vision intake system 132. The system captures high-resolution 3D video of the eye, allowing for detailed visualization of key anatomical structures such as the cornea, retina, and optic nerve. This data is operable to provide real-time feedback for both diagnostic purposes and treatment guidance. In alternative embodiments, the system may use different imaging devices, such as optical coherence tomography (OCT) or stereoscopic cameras, to capture the necessary visual data.

At step 1004, the system analyzes the captured 3D visual data to identify specific regions of the eye. Using computer vision algorithms, the system processes the visual data to detect and segment various anatomical structures, such as the cornea, lens, and retina. In one embodiment, the system may automatically identify the center of the pupil and track the position of the eye in real-time to ensure continuous focus on the region of interest. Additionally, the system may recognize other key features, such as the optic nerve or retinal layers, and use this information to guide subsequent steps in the process.

At step 1006, the system dynamically adjusts capture of the 3D visual data. For example, the system can adjust the focus and centering of the camera or slit-lamp based on the analyzed 3D visual data. Using servo motors or other mechanical controls, the system guides the camera to maintain precise focus on the identified region of the eye. For example, if the system detects that the eye has shifted or the focus has drifted, it automatically realigns the camera to ensure continuous clarity during the examination or treatment. In certain embodiments, the system may also provide visual cues or alerts to the user, notifying them of any adjustments being made. This automated focus adjustment ensures that the region of interest remains centered and in sharp focus throughout the procedure, minimizing the need for manual intervention.

At step 1008, the system retrieves relevant diagnostic and patient data to support the treatment process. The system queries diagnostic data datastore 135 and patient clinical data store 220 to retrieve medical information related to the region of the eye being examined. For example, the system may retrieve past measurements of corneal thickness, intraocular pressure data, or previous diagnostic assessments of retinal health. This information is presented alongside the real-time visual data, providing the doctor with a comprehensive view of the patient's condition during the procedure. In some embodiments, the system may use machine learning models to correlate the retrieved data with the current visual information, offering predictive insights or treatment recommendations based on historical trends.

At step 1010, the system provides real-time feedback to guide the treatment process based on computer vision analysis. During the treatment, such as laser therapy or other corrective procedures, the system continuously analyzes the 3D video feed to ensure that the treatment device remains centered on the region of interest. For example, if the system detects that the treatment device is veering off target, it automatically adjusts the positioning of the device using servo motors to maintain precision. Additionally, the system may analyze tissue response in real-time, providing immediate feedback on whether the treatment is proceeding as expected. In certain embodiments, the system may also prompt the user with corrective actions, such as suggesting focus adjustments or altering the treatment intensity based on real-time analysis of the eye's surface.

At step 1012, the system dynamically adjusts treatment parameters based on real-time analysis. In this step, the system uses machine learning models and computer vision techniques to continuously monitor the treatment process, adjusting parameters such as laser intensity, duration, or focus based on the feedback it receives from the visual data. For example, the system may reduce the laser intensity if it detects signs of excessive tissue damage or increase the duration of treatment if it identifies incomplete coverage of the targeted area. These real-time adjustments ensure that the treatment is both precise and effective, minimizing the risk of complications and improving patient outcomes.

At step 1014, the system integrates the visual data, diagnostic information, and treatment progress into a unified display for the user. The data presentation engine 210 organizes and presents this information in a mixed reality interface, allowing the doctor to see the real-time 3D visualization of the eye, overlaid with diagnostic data, treatment progress, and any system-suggested adjustments. For example, color-coded regions may indicate areas that have been successfully treated, while visual prompts may suggest further attention to untreated regions. The system ensures that all relevant information is presented clearly and interactively, allowing the user to make informed decisions throughout the procedure.

At step 1016, the system stores the visual data, treatment data, and diagnostic results in 3D eye data datastore 133 for future reference. The stored data includes the 3D video feed, any real-time adjustments made by the system, and diagnostic information gathered during the procedure. In future examinations, this data can be retrieved to assess the patient's progress or compare treatment outcomes. The system may also enable longitudinal analysis by comparing current data with past treatments, helping doctors to refine treatment strategies based on historical results.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

Any of the above-mentioned systems, units, modules, engines, controllers, interfaces, components, or the like may comprise hardware and/or software as described herein. For example, the system described in association with vision intake system 132, machine learning system 134, diagnostic data datastore 135, patient clinical data store 220, and subcomponents thereof may comprise computing hardware and/or software as described herein in association with the figures. Furthermore, any of the above-mentioned systems, units, modules, engines, controllers, interfaces, components, or the like may use and/or comprise an application programming interface (API) for communicating with other systems, units, modules, engines, controllers, interfaces, components, or the like for obtaining and/or providing data or information.

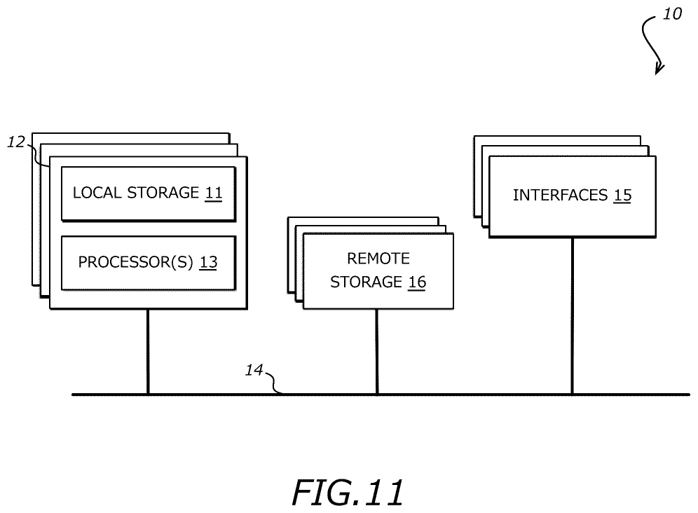

Referring now to FIG. 11, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software-or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 11 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

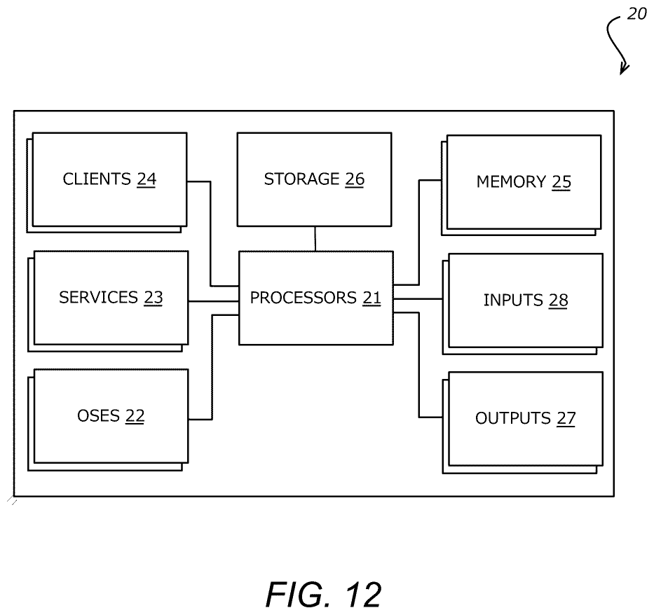

In some embodiments, systems may be implemented on a standalone computing system. Referring now to FIG. 12, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™, VisionOS, or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 11). Examples of storage devices 26 include flash memory, magnetic hard drive, CD-ROM, and/or the like.

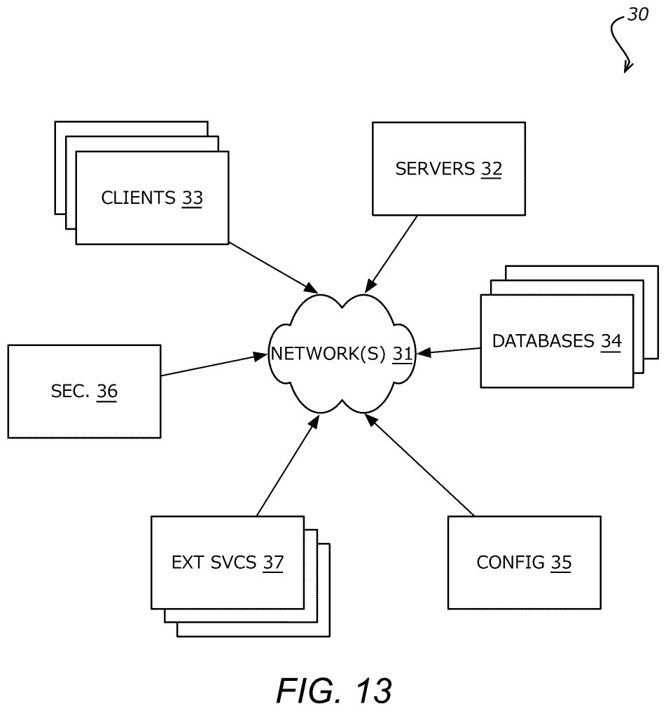

In some embodiments, systems may be implemented on a distributed computing network, such as one having any number of clients and/or servers. Referring now to FIG. 13, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 12. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

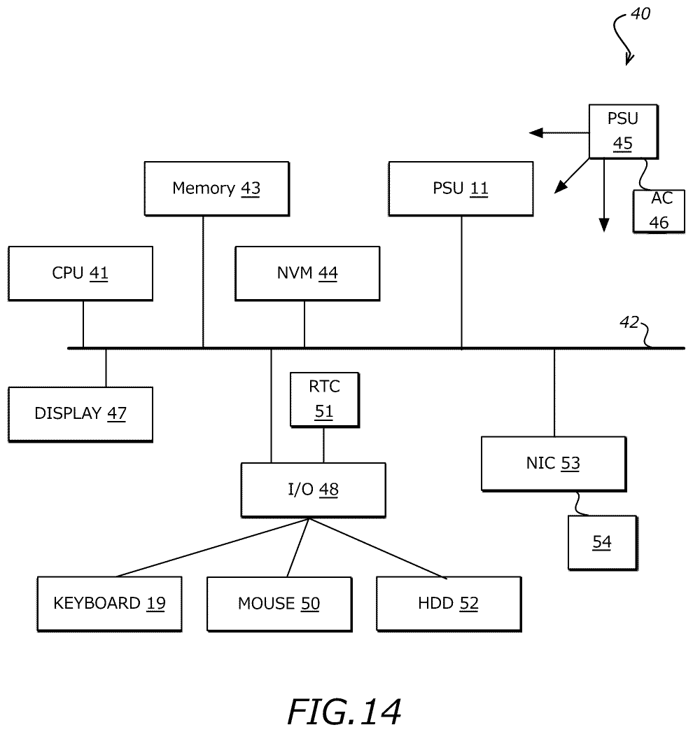

FIG. 14 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for facilitating database queries through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A computing system, comprising:
at least one computing processor; and
memory including instructions that, when executed by the at least one computing processor, enable the computing system to:
obtain three-dimensional (3D) data that includes a representation of an eye, the 3D data being captured in real-time and representing anatomical structures of the eye;
analyze the 3D data to identify at least one anatomical structure of the eye;
retrieve diagnostic data and patient-specific clinical information associated with the at least one anatomical structure of the eye;
integrate the 3D data with the diagnostic data and the patient-specific clinical information to generate an interactive visualization; and
present the interactive visualization via a mixed reality device, wherein the interactive visualization includes overlaid diagnostic information and user-controllable elements to manipulate diagnostic overlays.

2. The computing system of claim 1, wherein the 3D data is captured by a vision intake system comprising stereoscopic cameras.

3. The computing system of claim 1, wherein the computing system further determines a stage of an eye examination based on an anatomical structure of the eye identified in the 3D data.

4. The computing system of claim 3, wherein determining the stage of the eye examination further comprises applying computer vision algorithms to analyze the 3D data and detect specific examination phases by comparing changes in the anatomical structure of the eye to predefined stage models.

5. The computing system of claim 1, wherein the computing system applies machine learning models to the 3D data to detect potential abnormalities in the anatomical structures of the eye.

6. The computing system of claim 1, wherein a display system spatially organizes the diagnostic data within a mixed reality environment, wherein spatially organizing comprises overlaying relevant clinical information directly onto anatomical structures displayed in the 3D data.

7. The computing system of claim 1, further comprising a gesture-based input interface, wherein the gesture-based input interface allows a user to adjust the visualization.

8. The computing system of claim 1, wherein integration of the 3D data, diagnostic data, and patient-specific clinical information comprises automatically mapping diagnostic metrics and clinical records to identified anatomical structures in the 3D data using region-based tagging techniques.

9. The computing system of claim 8, wherein mapping is performed using landmark detection or region-based tagging techniques to associate diagnostic measurements, patient history, or clinical observations with corresponding regions of the 3D data.

10. The computing system of claim 1, wherein the interactive visualization includes dynamically generated overlays highlighting areas of the eye based on the diagnostic data.

11. A computer-implemented method, comprising:
obtaining three-dimensional (3D) data that includes a representation of an eye, the 3D data being captured in real-time and representing anatomical structures of the eye;
analyzing the 3D data to identify at least one anatomical structure of the eye;
retrieving diagnostic data and patient-specific clinical information associated with the at least one anatomical structure of the eye;
integrating the 3D data with the diagnostic data and the patient-specific clinical information to generate an interactive visualization; and
presenting the interactive visualization via a mixed reality device, wherein the interactive visualization includes overlaid diagnostic information and user-controllable elements to manipulate diagnostic overlays.

12. The computer-implemented method of claim 11, further comprising:
determining a stage of an eye examination based on an anatomical structure of the eye identified in the 3D data.

13. The computer-implemented method of claim 12, wherein determining the stage of the eye examination further comprises applying computer vision algorithms to analyze the 3D data and detect specific examination phases by comparing changes in the anatomical structure of the eye to predefined stage models.

14. The computer-implemented method of claim 11, further comprising:
applying machine learning models to the 3D data to detect potential abnormalities in the anatomical structures of the eye.

15. The computer-implemented method of claim 11, further comprising:
integrating the 3D data, diagnostic data, and patient-specific clinical information by automatically mapping diagnostic metrics and clinical records to identified anatomical structures in the 3D data using region-based tagging techniques.

16. The computer-implemented method of claim 11, further comprising:
spatially organizing the diagnostic data within a mixed reality environment, wherein spatially organizing comprises overlaying relevant clinical information directly onto anatomical structures displayed in the 3D data.

17. A non-transitory computer readable storage medium storing instructions that, when executed by at least one processor of a computing system, causes the computing system to:
obtain three-dimensional (3D) data that includes a representation of a an eye, the 3D data being captured in real-time and representing anatomical structures of the eye;
analyze the 3D data to identify at least one anatomical structure of the eye;
retrieve diagnostic data and patient-specific clinical information associated with the at least one anatomical structure of the eye;
integrate the 3D data with the diagnostic data and the patient-specific clinical information to generate an interactive visualization; and
present the interactive visualization via a mixed reality device, wherein the interactive visualization includes overlaid diagnostic information and user-controllable elements to manipulate diagnostic overlays.

18. The non-transitory computer readable storage medium of claim 17, wherein the instructions, when executed by the at least one processor, further enables the computing system to:
determining a stage of an eye examination based on an anatomical structure of the eye identified in the 3D data.

19. The non-transitory computer readable storage medium of claim 18, wherein the instructions, when executed by the at least one processor, further enables the computing system to:
apply computer vision algorithms to analyze the 3D data and detect specific examination phases by comparing changes in the anatomical structure of the eye to predefined stage models.

20. The non-transitory computer readable storage medium of claim 17, wherein the instructions, when executed by the at least one processor, further enables the computing system to:
apply machine learning models to the 3D data to detect potential abnormalities in the anatomical structures of the eye.
